(19) United States
(12) Patent Application Publication
(10) Pub. No.: US 2026/0071241 A1
(43) Pub. Date: Mar. 12, 2026

(54) BIOSYNTHESIS OF ISOPRENOID

(71) Applicant: Agency for Science, Technology and Research, Singapore (SG)

(72) Inventors: Congqiang Zhang, Singapore (SG); Nicola Yen Min (Yuanming) Tan (Chen), Singapore (SG); Xixian Chen, Singapore (SG); Sudha Devi D/O Manbahal Shukal, Singapore (SG); Jian Ting Leonard Ong, Singapore (SG); Chin Chin Lim, Singapore (SG)

(21) Appl. No.: 19/108,294

(22) PCT Filed: Nov. 10, 2023
(86) PCT No.: PCT/SG2023/050752

§ 371 Date: Mar. 3, 2025

(30) Foreign Application Priority Data
Nov. 14, 2022 (SG) 10202260093R

(51) Int. Cl. C12P 7/04; C12N 9/88

(52) U.S. Cl. CPC C12P 7/04; C12N 9/88; C12Y 402/03048

(57) ABSTRACT

There is provided a recombinant cell comprising a plasmid expressing terpenoid synthase gene and one or more modification to the gene of the recombinant cell. Also provided is a recombinant cell for use in biosynthesis and a method of producing isoprenoid/terpenoid.

### TECHNICAL FIELD

The present disclosure relates broadly to synthesis of isoprenoid/terpenoid. In particular, the present disclosure relates to a recombinant cell comprising a plasmid expressing terpenoid synthase/isoprenoid synthase gene and one or more modification to the gene of the recombinant cell and/or a method of producing isoprenoid/terpenoid.

### BACKGROUND

Isoprenoids, or terpenoids, have wide applications in food, feed, pharmaceutical, and cosmetic industries. Nerolidol, an acyclic C15 isoprenoid, also known as peruviol, is an important industrial molecule with wide range of applications. Nerolidol is widely used in cosmetics (e.g., perfumes and shampoos), in food products (as a flavouring agent and/or a food preservative), personal care products (e.g., detergents and cleansers), vitamin synthesis (e.g., vitamins E and K1), and as pharmaceutical chemicals (e.g., teprenone, α-sinensal, and 4-acetylantroquinonol B). Nerolidol also has various biological and pharmacological properties, such as anti-bacterial, antioxidant, anti-herbivore, anti-tumour, and anti-inflammatory.

The global usage of nerolidol per year is up to 100 metric tonnes. Currently, the commercial source of nerolidol is either plant extraction (e.g., from the flower of Cassia fistula, from the root of Oplopanax horridus (Sm.) Miq.) or chemical synthesis from linalool. Both methods are inefficient, costly and their products are low-quality as with many impurities. For chemical synthesis, the raw material is linalool, which is a non-renewable carbon source that is costly, less safe, and a synthetic flavour compound that is less favoured by consumers who are more aware of benefits of Environmental, Social, and Governance (ESG) and prefer green and sustainable products. Also, synthetic nerolidol contains both cis- and trans-isomers. Natural sources are mainly trans-nerolidol.

Many plants such as neroli, ginger, lemongrass, and lavender produce nerolidol naturally but is inefficient and costly as plants require long growth cycles and have very low nerolidol content/low yield. Furthermore, the nerolidol quality/purity varies from 0.4% to 80% according to different plants, harvest time, weather conditions, growing regions, and extraction methods. Extraction of nerolidol from plants may also be of low quality with many other impurities.

Therefore, there is a need to provide an alternative method to produce isoprenoid or terpenoids.

### SUMMARY

In one aspect, there is provided a recombinant cell comprising a plasmid expressing a terpenoid synthase gene and one or more modification to the gene of the recombinant cell.

In some examples, the terpenoid synthase is a sesquiterpenoid synthase.

In some examples, the sesquiterpenoid synthase is nerolidol synthase.

In some examples, the terpenoid synthase is selected from the group consisting of fungi, plants and/or bacteria.

In some examples, the terpenoid synthase is from any one selected from the group consisting of Fragaria ananassa (Fa)-strawberry, Vitis vinifera-grape, Selaginella moellendorffii (Sm)-Spikemoss, Celastrus angulatus Maxim, Maze Zea mays, Abrus precatorius, Acer yangbiense, Actinidia chinensis, Aegilops tauschii, Ageratina Adenophora, Albizia julibrissin, Ananas comosus, Antirrhinum majus, Apostasia shenzhenica, Aquilegia coerulea, Arabidopsis thaliana, Arabis alpine, Arachis duranensis, Arachis hypogaea, Artemisia annua, Beta vulgaris, Cajanus cajan, Camellia sinensis, Cannabis sativa, Capsicum annuum, Capsicum baccatum, Capsicum chinense, Carpinus fangiana, Castanea mollissima, Chengiopanax sciadophylloides, Chenopodium quinoa, Cicer arietinum, Citrus clementina, Citrus sinensis, Citrus unshiu, Coffea arabica, Coffea canephora, Coleus amboinicus, Coptis chinensis, Cucumis sativus, Cucurbita maxima, Cucurbita moschata, Davidia involucrate, Dorcoceras hygrometricum, Ensete ventricosum, Erythranthe guttata, Eucalyptus grandis, Fagus sylvatica, Ficus carica, Folsomia candida, Fragaria vesca, Glycine max, Glycine soja, Gossypium australe, Helianthus annuus, Herrania umbratical, Hevea brasiliensis, Hibiscus syriacus, Jasminum sambac, Jatropha curcas, Juglans regia, Kalanchoe fedtschenkoi, Kingdonia uniflora, Lactuca saligna, Lavandula angustifolia, Lupinus albus, Lupinus angustifolius, Malus baccata, Malus domestica, Manihot esculenta, Medicago sativa, Medicago truncatula, Microthlaspi erraticum, Mikania micrantha, Momordica charantia, Morella rubra, Morus notabilis, Mucuna pruriens, Nelumbo nucifera, Nicotiana attenuate, Nicotiana tabacum, Nyssa sinensis, Ocimum basilicum, Olea europaea subsp. Europaea, Oryza brachyantha, Panicum miliaceum, Phaseolus angularis, Phaseolus vulgaris, Phyllostachys edulis, Populus alba, Populus nigra, Populus trichocarpa, Prunus armeniaca, Prunus avium, Prunus campanulate, Prunus dulcis, Prunus persica, Punica granatum, Pyrus ussuriensis x Pyrus communis, Quercus lobata, Rhamnella rubrinervis, Rosa chinensis, Salix brachista, Salix dunnii, Salix suchowensis, Scoparia dulcis, Selaginella moellendorffii, Senna tora, Solanum lycopersicum, Spinacia oleracea, Striga asiatica, Tanacetum cinerariifolium, Tetracentron sinense, Theobroma cacao, Trifolium medium, Trifolium pratense, Trifolium subterraneum, Tripterygium wilfordii, Triticum Urartu, Vigna angularis var. angularis, Vigna radiata var. radiata, Zingiber officinale, and Ziziphus jujuba.

In some examples, the terpenoid synthase is a strawberry nerolidol synthase (FaLNS).

In some examples, the recombinant cell comprises one or more modifications to the gene of the recombinant cell, optionally the modification to the gene of the recombinant cell comprises one or more insertion and/or one or more deletion of the recombinant cell.

In some examples, the one or more modification to the gene of the recombinant cell comprises genes involved in the formation of by-products.

In some examples, the one or more modification to the gene of the recombinant cell comprises genes from the mixed acid fermentation pathway and/or lyase family.

In some examples, the modification to the gene of the recombinant cell is to one or more genes selected from the group consisting of lactose inhibitor (lacI), T7 RNA polymerase (T7RP), aroA, aroB, aroC, aroAB, aroBC, aroAC, aroABC, ackA, pta, ackA-pta, IdhA, pflB, poxB, adhE, adhE-pta, serC, frdABCD, eutD, adhE-IdhA, adhE-IdhA-pflB, adhE-IdhA-poxB, adhE-IdhA-ackA-pta, adhE-IdhA-poxB-pflB, poxB-pflB-ackA-pta, adhE-IdhA-poxB-pfIB-ackA-pta, tnaA, zapB, and combinations thereof.

In some examples, the one or more genes that are modified from the recombinant cell comprises the combination of ackA-pta, IdhA, pflB, poxB, and/or tnaA.

In some examples, the recombinant cell comprises an insertion of lactose inhibitor and/or T7 RNA polymerase between the ybhB and ybhC gene.

In some examples, the recombinant cell comprises a deletion of aroA, aroB, aroC, aroAB, aroBC, aroAC, aroABC, ackA, pta, ackA-pta, IdhA, pflB, poxB, adhE, adhE-pta, adhE-IdhA, adhE-IdhA-pflB, adhE-IdhA-poxB, adhE-IdhA-ackA-pta, adhE-IdhA-poxB-pflB, poxB-pflB-ackA-pta, adhE-IdhA-poxB-pflB-ackA-pta, tnaA, and/or zapB.

In some examples, the recombinant cell carries plasmids expressing genes of a biosynthetic pathway.

In some examples, the biosynthetic pathway comprises the nerolidol biosynthetic pathway and/or mevalonate pathway.

In some examples, the recombinant cell carries plasmid expressing genes of the nerolidol biosynthetic pathway and/or mevalonate pathway comprising hmgS, atoB, truncated HMG-CoA reductase (thmgR), mevk, pmk, pmd, and/or idi.

In some examples, the recombinant cell comprises one or more plasmids with a combination of strong or weak promoters.

In some examples, the recombinant cell comprises a combination of T7, TM1, TM2 and/or TM3 promoters.

In some examples, the recombinant cell comprises one or more modifications selected from the group consisting of
i) a deletion of ackA and pta,
ii) carrying plasmids expressing one or more genes selected from the group consisting of HMG-COA synthase (hmgS), acetoacetyl-CoA thiolase (atoB), HMG-CoA reductase (hmgR), aroC with T7, TM1, TM2 and/or TM3 promoter; mevalonate kinase (mevk), phosphomevalonate kinase (pmk), mevalonate pyrophosphate decarboxylase (pmd), IPP isomerase (idi), aroB with T7, TM1, TM2 and/or TM3 promoter; and/or
iii) nerolidol synthase (LNS), fpps (ispA), aroA with T7, TM1, TM2 and/or TM3 promoter;
iv) a deletion of IdhA,
v) carrying plasmids expressing hmgS, atoB, hmgR, aroC with T7, TM1, TM2 and/or TM3 promoter; mevk, pmk, pmd, idi, aroB with T7, TM1, TM2 and/or TM3 promoter; and/or
vi) LNS, ispA, aroA with T7, TM1, TM2, and/or TM3 promoter;
vii) a deletion of pflB,
viii) carrying plasmids expressing hmgS, atoB, hmgR, aroC with T7, TM1, TM2 and/or TM3 promoter; mevk, pmk, pmd, idi, aroB with T7, TM1, TM2 and/or TM3 promoter; and/or
ix) LNS, ispA, aroA with T7, TM1, TM2 and/or TM3 promoter;
x) a deletion of poxB,
xi) carrying plasmids expressing hmgS, atoB, hmgR, aroC with T7, TM1, TM2 and/or TM3 promoter; mevk, pmk, pmd, idi, aroB with T7, TM1, TM2 and/orTM3 promoter;
and/or
xii) LNS, ispA, aroA with T7, TM1, TM2 and/or TM3 promoter;
xiii) a deletion of tnaA,
xiv) carrying plasmids expressing hmgS, atoB, hmgR, aroC genes with T7, TM1, TM2 and/or TM3 promoter; mevK, pmk, pmd, idi, aroB genes with T7, TM1, TM2 and/or TM3 promoter; and/or
XV) LNS, ispA, aroA with T7, TM1, TM2 and/or TM3 promoter;
xvi) a deletion of adhE,
xvii) carrying plasmids expressing hmgS, atoB, hmgR, aroC genes with T7, TM1, TM2 and/or TM3 promoter; mevk, pmk, pmd, idi, aroB genes with T7, TM1, TM2 and/or TM3 promoter; and/or
xviii) LNS, ispA, aroA with T7, TM1, TM2 and/or TM3 promoter;
xix) a deletion of zapB,
xx) carrying plasmids expressing hmgS, atoB, hmgR, aroC genes with T7, TM1, TM2 and/or TM3 promoter; mevk, pmk, pmd, idi, aroB genes with T7, TM1, TM2 and/or TM3 promoter; and/or
xi) LNS, ispA, aroA with T7, TM1, TM2 and/or TM3 promoter.

In some examples, the recombinant cell is a microbial cell selected from the group consisting of a bacterial cell, a fungal cell, a plant cell, and an algae cell.

In some examples, the microbial cell is a bacterial cell, optionally the microbial cell is Escherichia coli.

In another aspect, there is provided a recombinant cell as disclosed herein for use in biosynthesis.

In yet another aspect, there is provided a method of producing isoprenoid, wherein the method comprises culturing the recombinant cell as disclosed herein.

In some examples, the method comprises culturing the recombinant cell in a medium comprising a carbon source with glucose, glycerol and/or lactose.

In some examples, the method comprises culturing the recombinant cell in a medium with an extractant that comprises a plant oil.

In some examples, the extractant comprises a sunflower oil.

In some examples, the method comprises culturing the recombinant cell with a bioprocess comprising fed-batch fermentation with single-phase fermentation and/or two-phase fermentation.

### DESCRIPTION OF EMBODIMENTS

Nerolidol, also known as peruviol, is a naturally occurring sesquiterpene alcohol with a pleasant floral odour, present in plants such as neroli, jasmine, lavender, and the like. In view of the many uses of nerolidol, there is a need to provide methods of producing nerolidol. The inventors of the present disclosure have discovered an alternative method of producing terpenoid/isoprenoid that provides improved purity and yield. The present disclosure provides for a method of producing isoprenoid/terpenoid that addressed at least one of the problems currently faced in the production of nerolidol.

As such, in one aspect, there is provided a recombinant cell comprising a plasmid expressing a terpenoid synthase/isoprenoid synthase gene and one or more modification to the gene of the recombinant cell.

As used herein, the term “recombinant cell” refers to a cell with DNA molecules formed by laboratory methods of genetic recombination (such as molecular cloning) that bring together genetic material from multiple sources, creating sequences that would not otherwise be found in the genome. In this case, the recombinant cell expresses a combination of genetic material from different species such as plant and microbe.

In some examples, there is provided a recombinant cell comprising a recombinant cell with a plasmid expressing a terpenoid synthase/isoprenoid synthase gene and one or more modification to the gene of the recombinant cell.

In some examples, there is provided a non-human organism comprising a plasmid expressing terpenoid synthase/isoprenoid synthase gene and one or more modification to the gene of the non-human organism.

As used herein, the term “isoprenoid” refers to any class of organic compounds composed of one or more units of hydrocarbons, with each unit consisting of five carbon atoms arranged in a specific pattern. Isoprenoid refers to any compound formally derived from one or more isoprene units. Examples of isoprenoid compounds produced by plants may include such as but is not limited to, menthol, ginkolids, steviol, camphor, curcuminoids, natural rubber, and the like.

As used herein, the term “terpenoid” or “terpenes” refers to a large and diverse class of naturally occurring organic chemicals derived from the 5-carbon compound isoprene, and the isoprene polymers called terpenes. Terpenoids are also known as isoprenoids. Terpenoid may include volatile compounds used in perfume and food flavours, turpentine, steroids, carotene pigments and rubber.

The terms “terpenes”, “terpenoids” and “isoprenoids” are used interchangeably.

In some examples, the non-human organism is a transgenic animal.

In some examples, the non-human organism may include but is not limited to, mice, rat, non-human primates, and the like. In some examples, the background of a mice may include but is not limited to, C57/BL6, BALB/c, CD-1, SCID, and the like. In some examples, the background of a rat may include but is not limited to, A/J, Sprague Dawley, Wistar, and the like. In some examples, non-human primates may include but is not limited to, Rhesus monkey, Japanese monkey, Olive baboon, Squirrel monkey, Capuchin monkey, and the like.

Metabolic engineering using microbes is an attractive alternative method to produce high-quality nerolidol at a low cost. Several teams have attempted to produce nerolidol using microbes. Combining inducible expression of the nerolidol pathway genes with bioprocess optimization enabled Saccharomyces cerevisiae to produce up to 5.5 g/L nerolidol in 6 days. In 2021, a study in the art identified two new nerolidol synthases from Celastrus angulatu and used them in S. cerevisiae to produce nerolidol. The nerolidol production was further enhanced by the overexpression of the mevalonate pathway genes and downregulation of ERG9 with HXT1 promoter. Recently, nerolidol production in Yarrowia lipolytica was improved by rational engineering of a plant nerolidol synthase and overexpression of carnitine acetyltransferase (CAT2) to increase the acetyl-CoA shuttling between peroxisome and cytosol. In addition to yeasts, Escherichia coli has been engineered to produce ˜324 mg/L nerolidol using the multidimensional heuristic process (MHP) method that systematically balances biosynthetic pathways with different promoters and ribosome-binding sites. Nerolidol synthase is the critical enzyme for nerolidol biosynthesis.

However, no study has been carried out on comparing nerolidol synthases from different organisms, and little has been explored on genomic editing of E. coli for nerolidol bioproduction. Particularly, several novel fungal linalool/nerolidol synthases have been discovered recently but not been applied in nerolidol biosynthesis. The inventors of the present disclosure developed a microbial synthesis route. Here, the inventors of the present disclosure first compared seven nerolidol synthases from three kingdoms (plants, fungi, and bacteria).

In some examples, the terpenoid synthase may comprise but is not limited to, monoterpenoid (such as linalool) synthase, sesquiterpenoid (such as nerolidol) synthase, diterpenoid synthase, caryophyllene synthase, guaiene synthase, santalene synthase, Selinene synthase, cadinene synthase, cardinol synthase, ledene/viridiflorene synthase, ledol/viridiflorol synthase, bisabolene synthase, farnesene synthase, bisabolol synthase, valencene synthase, patchoulol synthase, longipinene synthase, humulene synthase, santalol synthase, copaene synthase, cubebol synthase, cubenene synthase, cubenol synthase, epicubenol synthase, gurjunene synthase, germacrene synthase, elemene synthase, vetivenene synthase, muurolene synthase, muurolol synthase, amorphadiene synthase, protoilludane synthase, spirobicyclo[3.1.0] terpene synthase, and the like.

In some examples, the terpenoid synthase is a sesquiterpenoid synthase.

In some examples, the sesquiterpenoid synthase is nerolidol synthase.

In some examples, the terpenoid synthase is selected from the group consisting of fungi, plants and/or bacteria.

In some examples, the terpenoid synthase is from fungi (Agrocybe aegerita (Aa), Agrocybe pediades (Ap), Hypholoma sublateritium (Hp), Saccharomyces cerevisiae, Metarhizium robertsii, Phomopsis amygdali), plants (Fragaria ananassa (Fa)-strawberry, Vitis vinifera-grape, Selaginella moellendorffii (Sm)-Spikemoss, Celastrus angulatus Maxim, Maze Zea mays, Abrus precatorius, Acer yangbiense, Actinidia chinensis, Aegilops tauschii, Ageratina Adenophora, Albizia julibrissin, Ananas comosus, Antirrhinum majus, Apostasia shenzhenica, Aquilegia coerulea, Arabidopsis thaliana, Arabis alpine, Arachis duranensis, Arachis hypogaea, Artemisia annua, Beta vulgaris, Cajanus cajan, Camellia sinensis, Cannabis sativa, Capsicum annuum, Capsicum baccatum, Capsicum chinense, Carpinus fangiana, Castanea mollissima, Chengiopanax sciadophylloides, Chenopodium quinoa, Cicer arietinum, Citrus clementina, Citrus sinensis, Citrus unshiu, Coffea arabica, Coffea canephora, Coleus amboinicus, Coptis chinensis, Cucumis sativus, Cucurbita maxima, Cucurbita moschata, Davidia involucrate, Dorcoceras hygrometricum, Ensete ventricosum, Erythranthe guttata, Eucalyptus grandis, Fagus sylvatica, Ficus carica, Folsomia candida, Fragaria vesca, Glycine max, Glycine soja, Gossypium australe, Helianthus annuus, Herrania umbratical, Hevea brasiliensis, Hibiscus syriacus, Jasminum sambac, Jatropha curcas, Juglans regia, Kalanchoe fedtschenkoi, Kingdonia uniflora, Lactuca saligna, Lavandula angustifolia, Lupinus albus, Lupinus angustifolius, Malus baccata, Malus domestica, Manihot esculenta, Medicago sativa, Medicago truncatula, Microthlaspi erraticum, Mikania micrantha, Momordica charantia, Morella rubra, Morus notabilis, Mucuna pruriens, Nelumbo nucifera, Nicotiana attenuate, Nicotiana tabacum, Nyssa sinensis, Ocimum basilicum, Olea europaea subsp. Europaea, Oryza brachyantha, Panicum miliaceum, Phaseolus angularis, Phaseolus vulgaris, Phyllostachys edulis, Populus alba, Populus nigra, Populus trichocarpa, Prunus armeniaca, Prunus avium, Prunus campanulate, Prunus dulcis, Prunus persica, Punica granatum, Pyrus ussuriensis x Pyrus communis, Quercus lobata, Rhamnella rubrinervis, Rosa chinensis, Salix brachista, Salix dunnii, Salix suchowensis, Scoparia dulcis, Selaginella moellendorffii, Senna tora, Solanum lycopersicum, Spinacia oleracea, Striga asiatica, Tanacetum cinerariifolium, Tetracentron sinense, Theobroma cacao, Trifolium medium, Trifolium pratense, Trifolium subterraneum, Tripterygium wilfordii, Triticum Urartu, Vigna angularis var. angularis, Vigna radiata var. radiata, Zingiber officinale, Ziziphus jujuba), and/or bacteria (Streptomyces clavuligerus (Sc), Kitasatospora setae).

In some examples, the terpenoid synthase is from any one selected from the group consisting of Fragaria ananassa (Fa)-strawberry, Vitis vinifera-grape, Selaginella moellendorffii (Sm)-Spikemoss, Celastrus angulatus Maxim, Maze Zea mays, Abrus precatorius, Acer yangbiense, Actinidia chinensis, Aegilops tauschii, Ageratina Adenophora, Albizia julibrissin, Ananas comosus, Antirrhinum majus, Apostasia shenzhenica, Aquilegia coerulea, Arabidopsis thaliana, Arabis alpine, Arachis duranensis, Arachis hypogaea, Artemisia annua, Beta vulgaris, Cajanus cajan, Camellia sinensis, Cannabis sativa, Capsicum annuum, Capsicum baccatum, Capsicum chinense, Carpinus fangiana, Castanea mollissima, Chengiopanax sciadophylloides, Chenopodium quinoa, Cicer arietinum, Citrus clementina, Citrus sinensis, Citrus unshiu, Coffea arabica, Coffea canephora, Coleus amboinicus, Coptis chinensis, Cucumis sativus, Cucurbita maxima, Cucurbita moschata, Davidia involucrate, Dorcoceras hygrometricum, Ensete ventricosum, Erythranthe guttata, Eucalyptus grandis, Fagus sylvatica, Ficus carica, Folsomia candida, Fragaria vesca, Glycine max, Glycine soja, Gossypium australe, Helianthus annuus, Herrania umbratical, Hevea brasiliensis, Hibiscus syriacus, Jasminum sambac, Jatropha curcas, Juglans regia, Kalanchoe fedtschenkoi, Kingdonia uniflora, Lactuca saligna, Lavandula angustifolia, Lupinus albus, Lupinus angustifolius, Malus baccata, Malus domestica, Manihot esculenta, Medicago sativa, Medicago truncatula, Microthlaspi erraticum, Mikania micrantha, Momordica charantia, Morella rubra, Morus notabilis, Mucuna pruriens, Nelumbo nucifera, Nicotiana attenuate, Nicotiana tabacum, Nyssa sinensis, Ocimum basilicum, Olea europaea subsp. Europaea, Oryza brachyantha, Panicum miliaceum, Phaseolus angularis, Phaseolus vulgaris, Phyllostachys edulis, Populus alba, Populus nigra, Populus trichocarpa, Prunus armeniaca, Prunus avium, Prunus campanulate, Prunus dulcis, Prunus persica, Punica granatum, Pyrus ussuriensis x Pyrus communis, Quercus lobata, Rhamnella rubrinervis, Rosa chinensis, Salix brachista, Salix dunnii, Salix suchowensis, Scoparia dulcis, Selaginella moellendorffii, Senna tora, Solanum lycopersicum, Spinacia oleracea, Striga asiatica, Tanacetum cinerariifolium, Tetracentron sinense, Theobroma cacao, Trifolium medium, Trifolium pratense, Trifolium subterraneum, Tripterygium wilfordii, Triticum Urartu, Vigna angularis var. angularis, Vigna radiata var. radiata, Zingiber officinale, and Ziziphus jujuba.

In some examples, the terpenoid synthase is a strawberry nerolidol synthase (FaLNS).

The strawberry nerolidol synthase was the most active in Escherichia coli and was selected as the best nerolidol synthase for nerolidol bioproduction. The technology of the present invention is better and advanced than existing methods in the art that relies on microbial production by other enzymes.

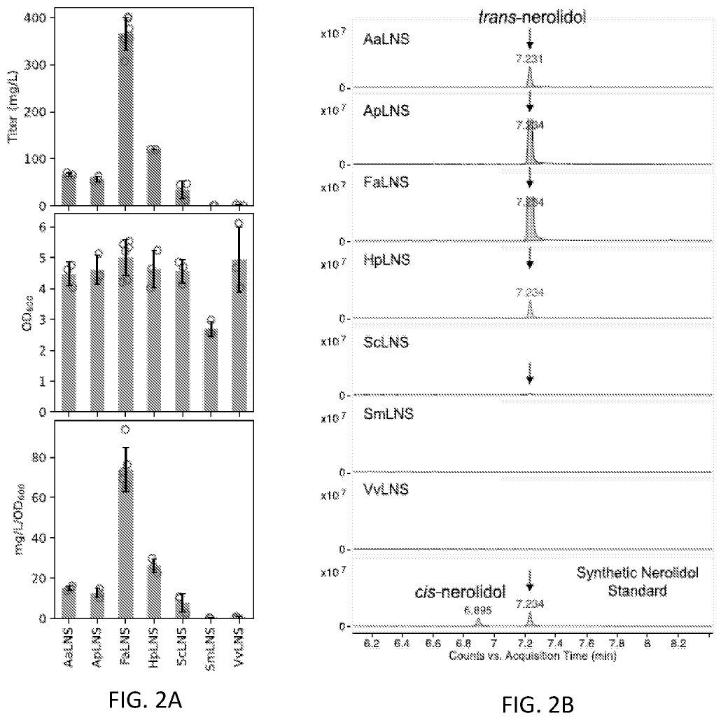

As shown in FIG. 2 of the present disclosure, the inventors of the present disclosure showed that the strawberry FaLNS produced the highest nerolidol titre of 366 mg/L, which was about 573-fold higher than that using SmLNS from Spikemoss (˜0.6 mg/L). The fungal LNSs (AaLNS, ApLNS, HpLNS) had higher activities than the bacterial LNS (ScLNS) but nerolidol yields are significantly lower (57-120 mg/L) than that of FaLNS.

In some examples, the modification to the gene of the recombinant cell may comprise, but is not limited to deletion, insertion, substitution, and/or translocation of one or more genes, and the like.

In some examples, the modification of genes comprises deletion of one or more genes.

In some examples, the deletion of genes may include but is not limited to one, or two, or three, or four genes, or five genes, or six genes, or seven genes, or eight genes, and the like.

In some examples, the recombinant cell comprises one or more modifications to the gene of the recombinant cell, optionally two or more modifications.

In some examples, the modification to the gene of the recombinant cell comprises one or more insertion and/or one or more deletion of the recombinant cell.

In some examples, the recombinant cell comprises one or more modifications to the gene of the recombinant cell, optionally the modification to the gene of the recombinant cell comprises one or more insertion and/or one or more deletion of the recombinant cell.

In some examples, the modification to the gene of the recombinant cell is to one or more genes such as, but is not limited to lacI, T7RP, aroA (5-enolpyruvylshikimate-3-phosphate synthase), aroB (3-dehydroquinate synthase), aroC (chorismate synthase), aroAB, aroBC, aroAC, aroABC, ackA, pta, ackA-pta, IdhA, pflB, poxB, adhE, adhE-pta, serC (phosphoserine aminotransferase), frdABCD (Fumarate reductase including four subunits: flavoprotein (frdA), iron-sulfur protein (frdB), and two hydrophobic anchor proteins (frdC and frdD)), eutD (Phosphate acetyltransferase), adhE-IdhA, adhE-IdhA-pfIB, adhE-IdhA-poxB, adhE-IdhA-ackA-pta, adhE-IdhA-poxB-pflB, poxB-pflB-ackA-pta, adhE-IdhA-poxB-pfIB-ackA-pta, tnaA, zapB, and combinations thereof.

In some examples, the recombinant cell comprises an insertion of lactose inhibitor and/or T7 RNA polymerase between the ybhB and ybhC gene.

In some examples, the recombinant cell comprises a deletion of aroA, aroB, aroC, aroAB, aroBC, aroAC, aroABC, ackA, pta, ackA-pta, IdhA, pflB, poxB, adhE, adhE-pta, adhE-IdhA, adhE-IdhA-pfIB, adhE-IdhA-poxB, adhE-IdhA-ackA-pta, adhE-IdhA-poxB-pfIB, poxB-pflB-ackA-pta, adhE-IdhA-poxB-pfIB-ackA-pta, tnaA, and/or zapB.

In some examples, the recombinant cell comprises an insertion of lactose inhibitor (lacI; lactose operon repressor) and/or T7 RNA polymerase (T7RP; T7 RNA polymerase) between the ybhB (putative kinase inhibitor) and ybhC (putative acyl-CoA thioester hydrolase) gene, and/or deletion of aromatic acids (aroABC; aroA (5-enolpyruvylshikimate-3-phosphate synthase), aroB (3-dehydroquinate synthase), aroC (chorismate synthase)), aroAB, aroBC, aroAC, aroABC, ackA, pta, ackA-pta, IdhA, pflB, poxB, adhE, adhE-pta, adhE-IdhA, adhE-IdhA-pflB, adhE-IdhA-poxB, adhE-IdhA-ackA-pta, adhE-IdhA-poxB-pflB, poxB-pfIB-ackA-pta, adhE-IdhA-poxB-pfIB-ackA-pta, tnaA, and/or zapB.

In some examples, the one or more modification to the gene of the recombinant cell comprises a gene involved in the formation of by-products.

In some examples, the one or more modification to the gene of the recombinant cell is a deletion of a gene involved in the formation of by-products.

In some examples, the by-products may include but is not limited to, acetate, formate, lactate, ethanol, succinate, CO₂, indole, ammonium, hydrogen sulfide, and the like, and combinations thereof.

In some examples, the one or more modification/deletion to the gene of the recombinant cell may comprise but is not limited to genes from the mixed acid fermentation pathway, lyase family, and the like.

In some examples, the one or more genes that are modified/deleted from the mixed acid fermentation pathway may include but is not limited to, acetate kinase (ackA), phosphate acetyltransferase (pta), acetate kinase-phosphate acetyltransferase (ackA-pta), D-lactate dehydrogenase (IdhA), formate acetyltransferase 1 (pflB), pyruvate dehydrogenase (poxB), aldehyde/alcohol dehydrogenase (adhE), adhE-pta, adhE-IdhA, adhE-IdhA-pfIB, adhE-IdhA-poxB, adhE-IdhA-ackA-pta, adhE-IdhA-poxB-pfIB, poxB-pflB-ackA-pta, adhE-IdhA-poxB-pflB-ackA-pta, and the like.

In some examples, the one or more genes that are modified/deleted from the recombinant cell may include genes from the lyase family. In some examples, the gene from the lyase family is the tryptophanase (tnaA).

In some examples, the one or more genes that are modified/deleted from the recombinant cell may include genes from a cell division factor. In some examples, the cell division factor is zapB.

In some examples, the one or more genes that are modified/deleted from the recombinant cell comprises the combination of ackA-pta, IdhA, pflB, poxB, and/or tnaA.

In some examples, the recombinant cell carries plasmids expressing genes of a biosynthetic pathway.

In some examples, the biosynthetic pathway may comprise but is not limited to the nerolidol biosynthetic pathway/mevalonate pathway, and the like.

In some examples, the recombinant cell carries plasmid expressing genes of the nerolidol biosynthetic pathway/mevalonate pathway such as but is not limited to, hmgS, atoB, truncated HMG-CoA reductase (thmgR), mevk, pmk, pmd, idi, and the like.

In some examples, the recombinant cell may comprise one or more plasmids with a combination of strong or weak promoters.

In some examples, the combination of promoters may include, but is not limited to T7 and its promoter variants (such as TM1, TM2 and/or TM3), araBAD, constitutive promoters, blue light promoters, thermopromoters, pTrc, CMV, EF-1α, CAG, OpIE1, OpIE2, and the like.

In some examples, the recombinant cell may comprise the combination of T7 and/or T7 promoter variants (such as TM1, TM2 and/or TM3). In some examples, the recombinant cell further comprises one or more modifications selected from the group consisting of
a deletion of ackA and pta,
carrying plasmids expressing one or more genes selected from the group consisting of HMG-COA synthase (hmgS), acetoacetyl-CoA thiolase (atoB), HMG-COA reductase (hmgR), aroC with T7 (and/or variants thereof such as TM1, TM2 and/or TM3) promoter; mevalonate kinase (mevk), phosphomevalonate kinase (pmk), mevalonate pyrophosphate decarboxylase (pmd), IPP isomerase (idi), aroB with T7 (and/or variants thereof such as TM1, TM2 and/or TM3) promoter; and/or
nerolidol synthase (LNS), fpps (ispA), aroA with T7 (and/or variants thereof such as TM1, TM2 and/or TM3) promoter.

In some examples, thmgR refers to the truncated version of hmgR. In some examples, thmgR has an amino acid sequence

MVLTNKTVISGSKVKSLSSAQSSSSGPSSSSEEDDSRDIESLDKKI
RPLEELEALLSSGNTKQLKNKEVAALVIHGKLPLYALEKKLGDTT
RAVAVRRKALSILAEAPVLASDRLPYKNYDYDRVFGACCENVIGY
MPLPVGVIGPLVIDGTSYHIPMATTEGCLVASAMRGCKAINAGGG
ATTVLTKDGMTRGPVVRFPTLKRSGACKIWLDSEEGQNAIKKAFN
STSRFARLQHIQTCLAGDLLFMRFRTTTGDAMGMNMISKGVEYSL
KQMVEEYGWEDMEVVSVSGNYCTDKKPAAINWIEGRGKSVVAEAT
IPGDVVRKVLKSDVSALVELNIAKNLVGSAMAGSVGGFNAHAANL
VTAVFLALGQDPAQNVESSNCITLMKEVDGDLRISVSMPSIEVGT
IGGGTVLEPQGAMLDLLGVRGPHATAPGTNARQLARIVACAVLAG
ELSLCAALAAGHLVQSHMTHNRKPAEPTKPNNLDATDINRLKDGS
VTCIKS*.

In some examples, the recombinant cell further comprises one or more modifications selected from the group consisting of
a deletion of IdhA,
carrying plasmids expressing hmgS, atoB, hmgR, aroC with T7 (and/or variants thereof such as TM1, TM2 and/or TM3) promoter; mevk, pmk, pmd, idi, aroB with T7 (and/or variants thereof such as TM1, TM2 and/or TM3) promoter; and/or
LNS, ispA, aroA with 77 (and/or variants thereof such as TM1, TM2 and/or TM3) promoter.

In some examples, the recombinant cell further comprises one or more modifications selected from the group consisting of
a deletion of pflB,
carrying plasmids expressing hmgS, atoB, hmgR, aroC with T7 (and/or variants thereof such as TM1, TM2 and/or TM3) promoter; mevk, pmk, pmd, idi, aroB with T7 (and/or variants thereof such as TM1, TM2 and/or TM3) promoter; and/or
LNS, ispA, aroA with T7 (and/or variants thereof such as TM1, TM2 and/or TM3) promoter.

In some examples, the recombinant cell further comprises one or more modifications selected from the group consisting of
a deletion of poxB,
carrying plasmids expressing hmgS, atoB, hmgR, aroC with T7 (and/or variants thereof such as TM1, TM2 and/or TM3) promoter; mevk, pmk, pmd, idi, aroB with T7 (and/or variants thereof such as TM1, TM2 and/or TM3) promoter; and/or
LNS, ispA, aroA with T7 (and/or variants thereof such as TM1, TM2 and/or TM3) promoter.

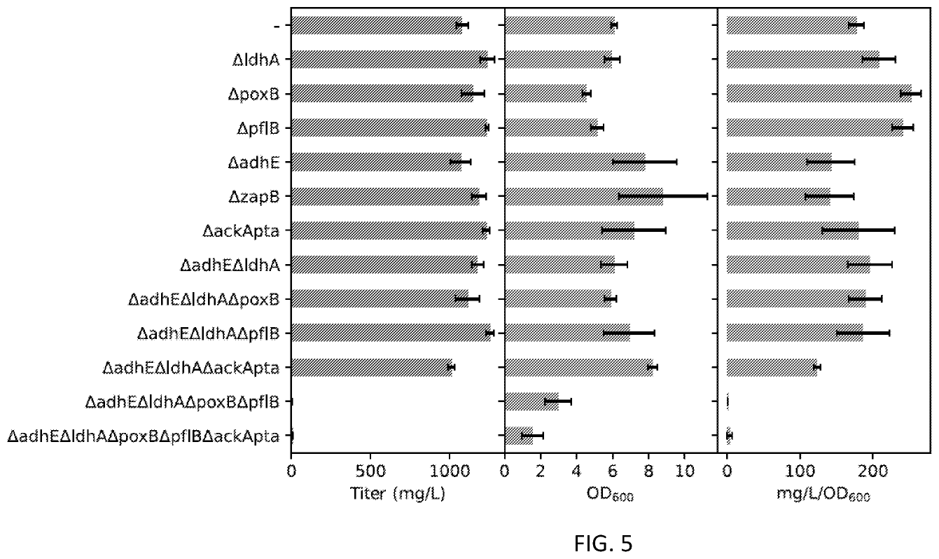

As shown in FIG. 5 in the present disclosure, the inventors of the present disclosure showed when grown in lactose media, deletion of IdhA, pflB, poxB, zapB, ackA-pta, and adhE-IdhA resulted in 15%, 14%, 6%, 10%, 14%, and 9% increase in nerolidol titres, respectively. The triple deletion strain (ΔadhEΔldhAΔpflB) increased nerolidol titre by 16%.

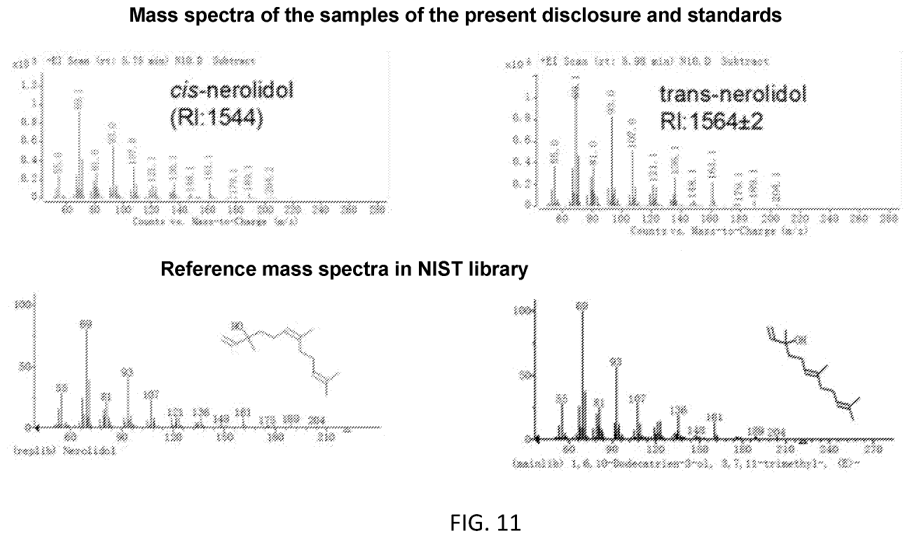

As shown in FIG. 12 of the present disclosure, the inventors of the present disclosure showed when grown in isopropyl β-D-1-thiogalactopyranoside (IPTG) media, the deletion of poxB and IdhA resulted in 12% and 7% increase in nerolidol production, respectively. The specific yields (mg/L/OD₆₀₀) of nerolidol were 18%, 8% and 14% higher in the poxB, pflB and IdIA deleted strain than that of the parental strain without deletion of the by-product genes.

In some examples, the recombinant cell further comprises one or more modifications selected from the group consisting of
a deletion of tnaA
carrying plasmids expressing hmgS, atoB, hmgR, aroC genes with T7 (and/or variants thereof such as TM1, TM2 and/or TM3) promoter; mevk, pmk, pmd, idi, aroB genes with T7 (and/or variants thereof such as TM1, TM2 and/or TM3) promoter; and/or
LNS, ispA, aroA with T7 (and/or variants thereof such as TM1, TM2 and/or TM3) promoter.

In some examples, the recombinant cell further comprises one or more modifications selected from the group consisting of
a deletion of adhE
carrying plasmids expressing hmgS, atoB, hmgR, aroC genes with T7 (and/or variants thereof such as TM1, TM2 and/or TM3) promoter; mevk, pmk, pmd, idi, aroB genes with T7 (and/or variants thereof such as TM1, TM2 and/or TM3) promoter; and/or
LNS, ispA, aroA with T7 (and/or variants thereof such as TM1, TM2 and/or TM3) promoter.

In some examples, the recombinant cell further comprises one or more modifications selected from the group consisting of
a deletion of zapB
carrying plasmids expressing hmgS, atoB, hmgR, aroC genes with T7 (and/or variants thereof such as TM1, TM2 and/or TM3) promoter; mevk, pmk, pmd, idi, aroB genes with T7 (and/or variants thereof such as TM1, TM2 and/or TM3) promoter; and/or
LNS, ispA, aroA with T7 (and/or variants thereof such as TM1, TM2 and/or TM3) promoter.

Through systematic optimization of the biosynthetic pathways, carbon sources, inducer, and genome editing, the inventors of the present disclosure constructed a series of deletion strains (single mutants ΔldhA, ΔpoxB, ΔpflB, and ΔtnaA; double mutants ΔadhE-ΔldhA; and triple mutants and beyond ΔadhE-ΔldhA-ΔpflB and ΔadhE-ΔldhA-ΔackA-pta) that produced high yields of 100% trans-nerolidol.

The strain of the present disclosure produced up to ˜16 g/L nerolidol in 5 L bioreactors in 3-4 days from low-cost feedstocks, e.g., glucose. The present disclosure's titres are about 53-fold and 1.45-2.9-fold higher than currently best achievement in E. coli and yeasts, respectively. Furthermore, the productivities of the present disclosure are 2.3-4.5 times higher than current highest achievements in the literature. The high yields, titres, and productivities of nerolidol in the present disclosure serve as a solid foundation for future commercial production of nerolidol. Beyond this study, the inventors of the present disclosure believe that the strategies of the present disclosure can inspire bioproduction of other valuable terpenoids.

In some examples, the recombinant cell may comprise, but is not limited to a mammalian cell, an insect cell, a microbial cell, and the like.

In some examples, the recombinant cell is a microbial cell including, but is not limited to, a bacterial cell, a fungal cell, a plant cell, an algae cell, and the like.

Examples of mammalian cells may include but is not limited to, human embryo kidney (HEK) cell, Chinese hamster ovary (CHO) cell, human cervical carcinoma (HeLa) cell, Vero cell, Baby Hamster Kidney (BHK) cell, and the like.

Examples of insect cells may include but is not limited to, Sf9 cells, S2 cells, Sf29 cells, and the like.

Examples of bacterial cells may include but is not limited to, Escherichia coli (E. coli), Bacillus, Corynebacterium, Lactic acid bacteria, Streptomyces, Cyanobacteria, and the like.

In some examples, the E. coli strain may include, but is not limited to, MG1655, BL21, DH5a, DH10B, W strain, Mach1, JM109 and the like.

Examples of fungal cells may include but is not limited to, Saccharomyces cerevisiae, Pichia pastoris, Yarrowia lipolytica, Rhodotorula toruloides, Aspergillus and the like.

Examples of plant cells may include but is not limited to, Lactuca sativa, Arabidopsis thaliana, Nicotiana tabacum, Nicotiana benthamiana, and the like.

Examples of algae cells may include but is not limited to, Chlamydomonas reinhardtii, Phaeodactylum tricornutum, and the like.

In some examples, the recombinant cell is a microbial cell. In various embodiments, the microbial cell is a bacterial cell. In some examples, the bacterial cell is Escherichia coli. In some examples, the Escherichia coli is MG1655.

Here, the inventors of the present disclosure systematically engineered Escherichia coli strain to produce trans-nerolidol as the main product. Subsequently, the inventors of the present disclosure systematically engineered E. coli strain through a series of pathway optimization (modular engineering strategy), genome editing (by blocking carbon flux from pyruvate and acetyl-CoA to byproducts), media (different carbon sources, C/N ratios, and inducers), and bioprocess (single-phase fermentation and two-phase extractive fermentation) optimization. The inventors of the present disclosure obtained a set of strains with high-nerolidol yields.

In another aspect, there is provided a recombinant cell for use in biosynthesis.

In another aspect, there is provided a method of producing isoprenoid/terpenoid, where in the method comprises culturing the recombinant cell of the present disclosure.

The method of the present disclosure includes a method that can produce trans-nerolidol in high purity, high content, high yield, and high productivity. The purity of trans-nerolidol can reach >95% with the strain of the present disclosure. The content can reach 400 g/kg of the cells, comparing to previously reported 0.2-1.3 g/kg of plant). The productivity is >4 g/L/day, about several-thousand times faster than plant synthesis. As compared to plant extraction, the method of the present disclosure is much faster (>1000 times), efficient and has significantly lower production cost. The present disclosure provides a green and cost-effective method of synthesizing trans-nerolidol using the raw material derived from renewable carbon sources (e.g., sugars and glycerol). As compared to chemical synthesis, the biological based method of the present disclosure is 100% trans-nerolidol, safer, more efficient and is generally more preferred by consumers. Compared to existing biotechnological route of nerolidol biosynthesis, the yields and titres of the present disclosure are 5-fold higher.

In some examples, the method comprises culturing the recombinant cell in a medium comprising one or more components including:
i) a carbon source
ii) an inducer
iii) an extractant
iv) a bioprocess with supplementation.

As used herein, an extractant refers to a liquid/solvent used in the extraction of a substance from a liquid/another material.

As used herein, a bioprocess refers to a specific process that uses complete living cells or their components (such as bacteria, enzymes, chloroplasts, viruses, cells, cell components) to obtain desired products. A bioprocess involves many reactions, both chemical and biochemical. It includes a method or operation of preparing a biological material, especially a product of genetic engineering for commercial use.

In some examples, the carbon source may comprise but is not limited to, glucose, glycerol, lactose, xylose, arabinose, acetate, methanol, ethanol, formate, fructose, sucrose, maltose, and the like.

In some examples, the method comprises culturing the recombinant cell in a medium comprising a carbon source with glucose, glycerol and/or lactose.

In some examples, the carbon source is glucose, glycerol and/or lactose.

As used herein, the term “medium” refers to a growth medium suitable for the culture of recombinant cells/host cells/non-human organisms including microbial or mammalian cells, of which all the chemical components in the medium are known.

In some examples, the inducer includes, but is not limited to, isopropyl β-D-1 thiogalactopyranoside (IPTG), lactose, galactose and the like.

In some examples, the carbon source and inducer may include but is not limited to, IPTG induced with glucose only, lactose induced with glucose, glycerol and lactose, and the like.

In some examples, the glucose concentration in the medium may include the range of 0.1 g/L to 20 g/L. In some examples, the glucose concentration in the medium may include 0.1 g/L, 0.5 g/L, 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, and the like.

In some examples, the glycerol concentration in the medium may include the range of 0.1 g/L to 30 g/L. In some examples, the glucose concentration in the medium may include 0.1 g/L, 0.5 g/L, 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, and the like.

In some examples, the lactose concentration in the medium may include the range of 1 mM to 30 mM. In some examples, the lactose concentration in the medium may include 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM and the like.

In some examples, the medium comprises an IPTG induced media containing only 10 g/L glucose.

In some examples, the medium comprises a lactose induced media containing 1-2 g/L glucose, 8-20 g/L glycerol and 15-30 mM lactose.

In some examples, the method comprises culturing the recombinant cell in a medium with an extractant that comprises a plant oil.

In some examples, the extractant comprises a plant oil.

In some examples, the plant oil may include, but is not limited to sunflower oil, dodecane, soybean oil, palm oil, coconut oil, peanut oil, canola oil, and the like.

In some examples, the extractant comprises the sunflower oil or dodecane.

In some examples, the extract comprises sunflower oil.

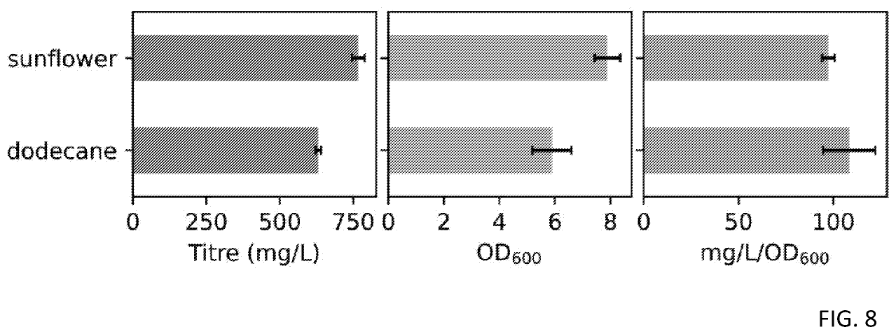

As shown in FIG. 8 of the present disclosure, the inventors of the present disclosure showed that the use of sunflower oil led to 20% to 34% higher nerolidol production and biomass than that of dodecane, respectively. Without wishing to be abound by theory, the benefit could be attributed to the boosting effect of nutrients such as vitamins, fatty acids, trace elements in the sunflower oil on bacterial growth.

In some examples, the bioprocess may comprise but is not limited to batch fermentation, fed-batch fermentation, continuous fermentation, and the like.

In some examples, the bioprocess comprises fed-batch fermentation.

In some examples, the fed-batch fermentation may comprise single-phase fermentation and/or two-phase fermentation.

In some examples, the method comprises culturing the recombinant cell with a bioprocess comprising fed-batch fermentation with single-phase fermentation and/or two-phase fermentation.

In some examples, the fed-batch fermentation (such as two-phase fermentation) may comprise supplementation of dodecane.

In some examples, the two-phase fermentation may include supplementation of 0.5% to 40% of dodecane. In some examples, the two-phase fermentation may include supplementation of 0.5%, 1%, 5%, 10%, 15%, 20%, 25%, 30%, 35%, or 40% of dodecane.

The inventors of the present disclosure showed that about 16 g/L nerolidol is produced within 4 days with about 9% carbon yield (g/g) with the two-phase fermentation, and >6.8 g/L nerolidol is produced in 3 days with the single-phase fermentation with low-cost renewable carbon sources (such as sugars and glycerol). To the best of the knowledge of the inventors of the present disclosure, the titres and productivity of the present disclosure are the highest in the literature, paving the way for future commercialization and inspiring biosynthesis of other isoprenoids.

Also disclosed are recombinant cells/products/methods as described herein.

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” or “attached” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “adjacent” used herein when referring to two elements refers to one element being in close proximity to another element and may be but is not limited to the elements contacting each other or may further include the elements being separated by one or more further elements disposed therebetween. For example, the cleavage compound as described herein cleaves the oligonucleotide (e.g., primer, probe, and the like) within or adjacent to the cleavage domain. Thus, the term “adjacent” means that the cleavage compound cleaves the oligonucleotide at either the 5′-end or the 3′ end of the cleavage domain. In some examples of the present disclosure, the cleavage reactions yield a 5′-phosphate group and a 3′-OH group.

The term “and/or”, e.g., “X and/or Y” is understood to mean either “X and Y” or “X or Y” and should be taken to provide explicit support for both meanings or for either meaning. Further, in the description herein, the word “substantially” whenever used is understood to include, but not restricted to, “entirely” or “completely” and the like. As used herein, the term “substantially no” or “very low” refers to a sequence homology of less than at least 20%, or 19%, or 18%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.9%, or 0.8%, or 0.7%, or 0.6%, or 0.5%, or 0.4%, or 0.3%, or 0.2%, or 0.1%, or 0.01% sequence homology to the target nucleic acid (for example any human gene). In some examples, the term “substantially no” or “very low” sequence homology refers to the control gene having substantially different sequence to the target nucleic acid (for example any human gene). In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### EXPERIMENTAL SECTION

### Methods

### Strain and Plasmid Construction

E. coli MG1655 strain (CGSC #6300) from the Coli Genetic Stock Center (CGSC) of Yale university was used in this study. T7 RNA polymerase gene was amplified from BL21 DE3 strain and inserted in the genome of MG1655 strain, located between the ybhB and ybhC gene. The resulting strain was named MGT7 (Table 3). Subsequently, the 3 genes aroABC were deleted sequentially from the MGT7 strain, and the resulting strain was named as MT7a1 (Table 3). On top of MT7a1, tnaA, pflB, poxB, adhE, IdhA, zapB and ackA-ptaA were deleted in various combinations to generate multi-gene-deletion mutant strains (Table 3) by the CRISPR-Cas9 method as previously described by studies in the art.

The three plasmids [p15Akan-LNS-ispA, p15A-spec-hmgS-atoB-hmgR (L2-8), and p15A-cammevk-pmk-pmd-idi (L2-5)] and their derivatives were designed as previously described by studies in the art. Various codon-optimized linalool/nerolidol synthases (LNSs, listed in Table 1) were synthesized by Integrated DNA Technologies (Singapore) and inserted into the operon of the plasmids p15A-kan-EBIA by replacing EBI with LNS. The plasmid details are listed in Table 2.

E. coli MG1655 strain (CGSC #6300) from the Coli Genetic Stock Center (CGSC) of Yale university was used in this study. T7 RNA polymerase gene was amplified from BL21 DE3 strain and inserted in the genome of MG1655 strain, located between the ybhB and ybhC gene. The resulting strain was named MT7 (Table 3). Subsequently, the three genes aroABC were deleted sequentially from the MT7 strain to create an auxotrophic system with our plasmids as previously described. The resulting strain was named MT7a1 (Table 3). On top of MT7a1, tnaA, pflB, poxB, adhE, IdhA, zapB, and ackA-ptaA were deleted in various combinations to generate single or multi-gene deletion mutant strains (Table 3) by the CRISPR-Cas9 method as previously described by studies in the art.

TABLE 1
Nerolidol Synthase Information

| | uniprot | | Length | Kₘ | K_{cat} | | |
|---|---|---|---|---|---|---|---|
| no | ID | name | (aa) | (μm) | (min⁻¹) | hosts | kingdom |
| 1 | | AaLNS | 384 | 9.0 | 3.3 | Agrocybe | fungi |
| | | | | | | aegerita | |
| 2 | | ApLNS | 343 | | | Agrocybe | fungi |
| | | | | | | pediades | |
| 3 | | HpLNS | 344 | | | Hypholoma | fungi |
| | | | | | | sublateritium | |
| | | | | | | (brick cap) | |
| 4 | P0CV94 | FaLNS | 519 | 8.1 | 3 | Fragaria | plants |
| | | | | | | ananassa | |
| | | | | | | (strawberry) | |
| 5 | H6WBC5 | VvLNS | 577 | | | Vitis vinifera | plants |
| | | | | | | (grape) | |
| 6 | D8RNZ9 | SmLNS | 368 | | | Selaginella | plants |
| | | | | | | moellendorffii | |
| | | | | | | (spikemoss) | |
| 7 | B5H117 | ScLNS | 333 | 9.6 | 18.7 | Streptomyces | bacteria |
| | | | | | | clavuligerus | |

TABLE 2
Plasmid summary

| Name | Plasmid information | Remarks |
|---|---|---|
| TM1HAT | p15A-TM1-spec-aroC- | Expressing three genes in the module 1 or HAT |
| | hmgS-atoB-hmgR (L2-8) ¹ | and aroC gene |
| TM2HAT | p15A-TM2-spec-aroC- | Expressing three genes in the module 1 or HAT |
| | hmgS-atoB-hmgR (L2-8) ¹ | and aroC gene |
| TM3HAT | p15A-TM3-spec-aroC- | Expressing three genes in the module 1 or HAT |
| | hmgS-atoB-hmgR (L2-8) ¹ | and aroC gene |
| TM1MPPI | p15A-TM1-cam-aroB- | Expressing three genes in the module 2 or MPPI |
| | mevK-pmk-pmd-idi (L2-5) ¹ | and aroB gene |
| TM2MPPI | p15A-TM2-cam-aroB- | Expressing three genes in the module 2 or MPPI |
| | mevK-pmk-pmd-idi (L2-5) ¹ | and aroB gene |
| TM3MPPI | p15A-TM3-cam-aroB- | Expressing three genes in the module 2 or MPPI |
| | mevK-pmk- pmd-idi(L2-5) ¹ | and aroB gene |
| LI | p15A-kan-aroA-LNS- | Expressing three genes in the module 3 or NI and |
| | ispA² | aroA gene |

¹ Here, TM1, TM2 and TM3 refer 3 different T7 promoter variants, whose relative strength was about 92%, 31% and 16% as compared to that of the wildtype T7 promoter¹.
²Various LNSs (Table 1) were used here.

TABLE 3
Strain summary

| Strain | | |
|---|---|---|
| name | Genetic background | Remarks |
| MG1655 | MG1655 | CGSC#6300 from the Coli Genetic Stock Center. |
| MT7 | MG1655-T7RP⁺ | The genes lacl and T7RP were knocked in the |
| | | genome of MG1655 between the ybhB and ybhC gene. |
| MT7a1 | MGT7ΔaroABC | The three genes aroABC were deleted from the |
| | | genome of MGT7. |
| MT7a11 | MT7a1ΔtnaA | The gene tnaA was deleted from the genome of MT7a1. |
| | MT7a1ΔackA-pta | The genes ackA and pta were deleted from the |
| | | genome of MT7a1. |
| | MT7a1ΔadhE | The gene adhE was deleted from the genome of MT7a1. |
| | MT7a1ΔldhA | The gene ldhA was deleted from the genome of MT7a1. |
| | MT7a1ΔpflB | The gene pflB was deleted from the genome of MT7a1. |
| | MT7a1ΔpoxB | The gene poxB was deleted from the genome of MT7a1. |
| | MT7a1ΔzapB | The gene zapB was deleted from the genome of MT7a1. |
| MT7b1 | MT7a1ΔadhEΔldhA | The genes adhE and ldhA were deleted from the |
| | | genome of MT7a1. |
| | MT7b1ΔpflB | The gene pflB was deleted from the genome of MT7b1. |
| | MT7b1ΔpoxB | The gene poxB was deleted from the genome of MT7b1. |
| | MT7b1ΔackA-pta | The genes ackA and pta were deleted from the |
| | | genome of MT7b1. |
| | MT7b1ΔpoxBΔpfIBΔackA-pta | The genes poxB, pflB, ackA and pta were deleted |
| | | from the genome of MT7b1. |
| #111 | MT7a1, TM1HAT, TM1MPPI | MT7a1 strain carrying the 3 plasmids TM1HAT, |
| | and LI | TM1MPPI and LI |
| #121 | MT7a1, TM1HAT, TM2MPPI | MT7a1 strain carrying the 3 plasmids TM1HAT, |
| | and LI | TM2MPPI and LI |
| #131 | MT7a1, TM1HAT, TM3MPPI | MT7a1 strain carrying the 3 plasmids TM1HAT, |
| | and LI | TM3MPPI and LI |
| #221 | MT7a1, TM2HAT, TM2MPPI | MT7a1 strain carrying the 3 plasmids TM2HAT, |
| | and LI | TM2MPPI and LI |
| #231 | MT7a1, TM2HAT, TM3MPPI | MT7a1 strain carrying the 3 plasmids TM2HAT, |
| | and LI | TM3MPPI and LI |
| #311 | MT7a1, TM3HAT, TM1MPPI | MT7a1 strain carrying the 3 plasmids TM3HAT, |
| | and LI | TM1MPPI and LI |
| #331 | MT7a1, TM3HAT, TM3MPPI | MT7a1 strain carrying the 3 plasmids TM3HAT, |
| | and LI | TM3MPPI and LI |
| #211 | MT7a1, TM2HAT, TM1MPPI | MT7a1 strain carrying the 3 plasmids TM2HAT, |
| | and LI | TM1MPPI and LI |
| #321 | MT7a1, TM3HAT, TM2MPPI | MT7a1 strain carrying the 3 plasmids TM3HAT, |
| | and LI | TM2MPPI and LI |
| #211a¹ | #211ΔtnaA | MT7a11 strain carrying the 3 plasmids TM3HAT, |
| | | TM2MPPI and LI |
| #211b¹ | #211ΔackA-pta | MT7a1ΔackA-pta strain carrying the 3 plasmids |
| | | TM2HAT, TM1MPPI and LI. |
| #211c | #211ΔadhE | MT7a1ΔadhE strain carrying the 3 plasmids |
| | | TM2HAT, TM1MPPI and LI. |
| #211d¹ | #211ΔldhA | MT7a1ΔldhA strain carrying the 3 plasmids |
| | | TM2HAT, TM1MPPI and LI. |
| #211e¹ | #211ΔpflB | MT7a1ΔpflB strain carrying the 3 plasmids |
| | | TM2HAT, TM1MPPI and LI. |
| #211f¹ | #211ΔpoxB | MT7a1ΔpoxB strain carrying the 3 plasmids |
| | | TM2HAT, TM1MPPI and LI. |
| #211g | #211ΔzapB | MT7a1ΔzapB strain carrying the 3 plasmids |
| | | TM2HAT, TM1MPPI and LI. |

¹Here, TM1, TM2 and TM3 refer 3 different T7 promoter variants, whose relative strength was about 92%, 31% and 16% as compared to that of the wildtype T7 promoter¹.

### Small-Scale Terpene Production

Chemically defined medium (CDM) consisted of 10 mL/L trace-element stock solution, 2 g/L (NH4)2SO4, 4.2 g/L KH2PO4, 11.24 g/L K2HPO4, 1.7 g/L citric acid, and 0.5 g/L MgSO4, pH 6.8-6.9. Two types of carbon sources were used: (1) type 1, 10-12.7 g/L glucose; (2) type 2, 2 g/L glucose, 8 g/L glycerol, and 10-50 mM lactose. The trace-element stock solution (100×) contained 0.25 g/L CoCl2·6H2O, 1.5 g/L MnSO4·4H2O, 0.8 g/L Zn (CH3COO) 2, 0.15 g/L CuSO4·2H2O, 0.3 g/L H3BO3, 0.84 g/L EDTA, 0.25 g/L Na2MoO4·2H2O, and 5 g/L Fe (III) citrate, and pH 8.0. For bioproduction, the cells were grown in a 14 mL BD Falcon tube with 1 mL of CDM or 10 mL of CDM in a 125 mL baffled Pyrex flask at 28° C./300 rpm for 3 days. The cells were first grown at 37° C./300 rpm until the OD₆₀₀ reached 0.8-1, thereafter, induced with 0.01-0.25 mM isopropyl β-D-1-thiogalactopyranoside (IPTG) (type 1) or automatically induced by 15 mM lactose (type 2) and were subsequently grown at 28° C. for 3 days. The antibiotics (34 μg/mL chloramphenicol, 50 g/mL spectinomycin, and 50 μg/mL kanamycin) were added in the culture to retain the three plasmids.

### Extraction and Quantification of Nerolidol

Nerolidol products were extracted from cells, supernatant, and organic layer. Samples from two-phase fermentation were prepared in a dilution range of 1000-20,000× by diluting the organic layer into hexane. Supernatant and cell pellet were separated from single-phase fermentation by centrifugation at 13,000 g for 10 min. The aqueous phase (supernatant) was extracted by vortexing at a ratio of 10:1 of hexane: supernatant for 30 min. Following centrifugation, the hexane layer from the mixture was collected and diluted by 50×, which was subsequently used for the measurement of nerolidol representative of the aqueous phase. Intracellular nerolidol was extracted from cell pellets by adding 1 mL of the hexane, acetone, and ethanol (HAE) mixture in a 2:1:1 ratio. The homogeneous solution was vortexed at 50° C. for 30 min and another 30 min at room temperature. Subsequently, the mixture was centrifuged, and the HAE layer was collected and diluted 100× for further quantification. The prepared samples were then analysed on an Agilent 7890 gas chromatograph equipped with an Agilent 5977B MSD. Samples were injected into the Agilent VF-WAXms column with a split ratio of 40:1 at 240° C. The oven program started at 100° C. for 1 min, was raised up to 150° C. at 50° C./min, then to 240° C. at 15° C./min, and maintained at 240° C. for another 2 min. The sample concentrations were calculated by interpolating with a commercial Nerolidol standard curve purchased from Sigma-Aldrich (Cat #00459-1ML). A mass spectrometer was operated in the electrospray ionization (EI) mode with full scan analysis (m/z 30-300, 2 spectra/s).

### Bioreactor Fermentation for Nerolidol Production

Two 7 L Bioreactors (Applikon Biotechnology) with a 5 L working volume were used in the study of the present disclosure. The cells (−80° C. stock) were grown in 50 mL of CDM for 16 h at 37° C. in a 250 mL shake flask. The preculture of approximately OD₆₀₀ of 5 was then harvested, washed, and inoculated into fresh 1.8 L of CDM in the bioreactor. After 9-10 h, feed mixture (5 g/L MgSO4 and 500 g/L glucose) was added gradually into the bioreactor at the rate of 9.2-56 mL/h for 10 h until OD₆₀₀ reached about 50-60, when cells were induced by 0.1 mM IPTG. After induction, the feeding rate was kept constant at 40 mL/h for about 20 h and subsequently reduced to about 30 mL/h till completion (˜72 h). Also, the temperature was then adjusted and maintained at 30° C. For two-phase fermentation, 1/5 (v/v) of dodecane was supplemented in the bioreactor. During fermentation, the dissolved oxygen level was sustained at 30% (800-1300 rpm) by supplying filtered air at a gas rate of 0.13-1.75 vvm. A 28% ammonia solution was used to maintain a culture pH of 7.0.

### Results

### Pathway Design and Enzyme Screening

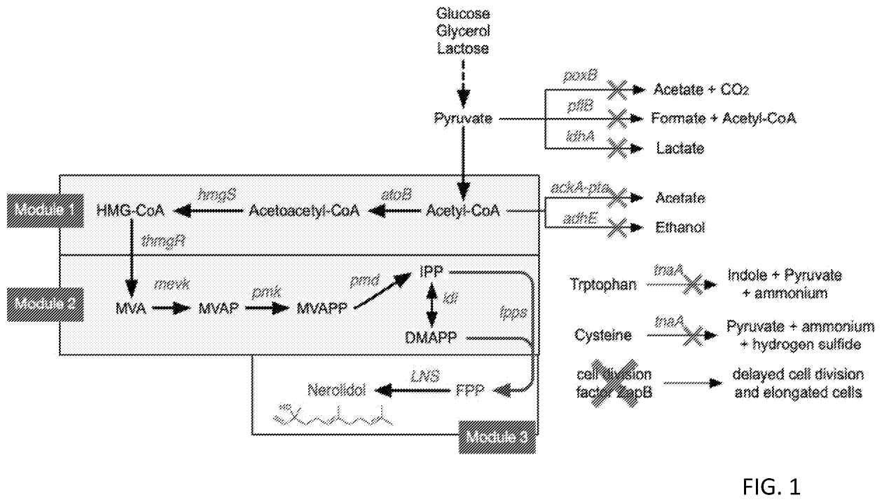

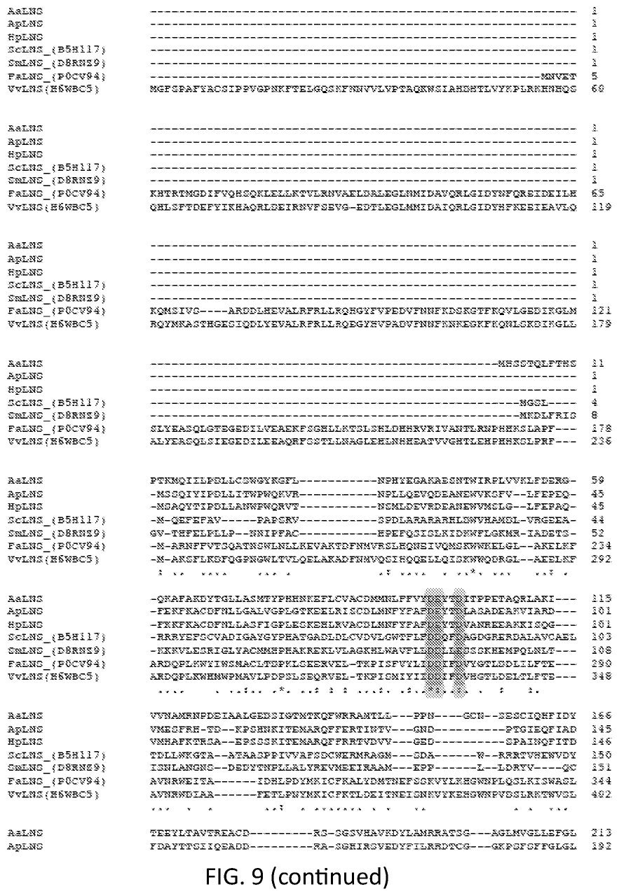

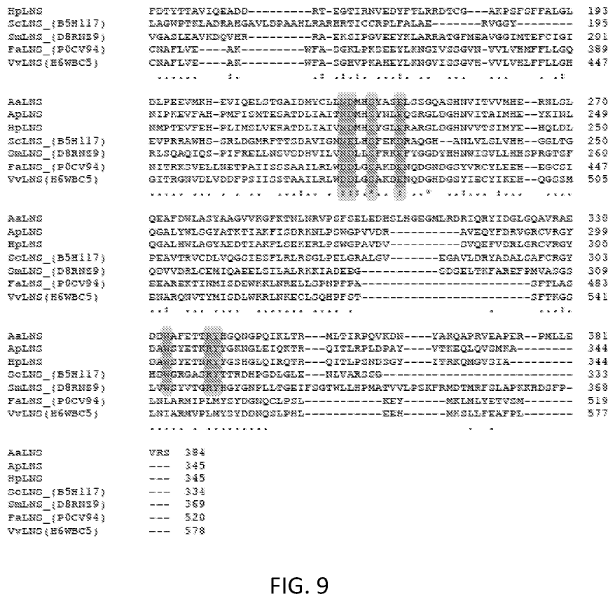

The inventors of the present disclosure first designed the nerolidol pathway with a multidimensional heuristic process (MHP) method. Briefly, the whole biosynthetic pathway was segmented into three modules: module 1 with the three enzymes HmgS, AtoB, and the truncated HmgR (tHmgR); module 2 with four enzymes MevK, Pmk, Pmd, and Idi; and module 3 with two enzymes Fpps (or ispA) and linalool/nerolidol synthase (LNS) (FIG. 1). Each module was controlled by T7 promoter variants (TM1/2/3). Except LNS, all the other genes were the same as previously described by studies in the art. For LNS, the inventors of the present disclosure have selected seven LNSs from different organisms covering fungi, plants, and bacteria (Table 1). Based on the sequence alignment in (FIG. 9), the inventors of the present disclosure observed that the two plant LNSs (FaLNS and VvLNS) have about 63% identity (FIG. 10A); the three fungal LNSs (AaLNS, ApLNS, and HpLNS) have about 34-36% identity; and the Spikemoss LNS (SmLNS) is more closely related to the bacterial ScLNS, with about 23% identity, than that to FaLNS and VvLNS. The phylogenetic tree is provided in FIG. 10B. Although the two plant LNSs (FaLNS and VvLNS) are noticeably larger than the other five LNSs, the two metal-binding motifs, DDxxD and NDxxSxxxE, are highly conserved among all the seven LNSs, with Asp replaced with Glu in certain cases (FIG. 9). Also, the active-site motif WxxxxxRY is conserved in microbial LNSs but not in plant LNS (except SmLNS).

Three out of the seven LNSs are characterized in vitro, with kinetic parameters summarized in Table 1. Among them, Kₘ values are similar, but ScLNS has a relatively higher Kcat value than FaLNS and AaLNS. However, in vitro kinetics are not always consistent with in vivo kinetics due to expression and solubility differences. Hence, the inventors of the present disclosure compared them by experiments. FIGS. 2A and 2B shows the screening of different nerolidol synthases. Among the seven LNSs, FaLNS was most active, and its expressing strain produced the highest amount of nerolidol, 366 mg/L (FIG. 2A), which was about 573-fold higher than that using SmLNS and VvLNS (˜0.6 mg/L). The fungal LNSs (AaLNS, ApLNS, and HpLNS) had higher activities than the bacterial one (ScLNS) but the nerolidol yields (57-120 mg/L) of their expressing strains were 67-84% lower than that of FaLNS-expressing strain. The biomass (OD₆₀₀) of different strains was similar, except for the one expressing SmLNS was relatively lower (FIG. 2A). Hence, the specific yields (mg/L/OD₆₀₀, FIG. 2A) had the same trend as that of the titres.

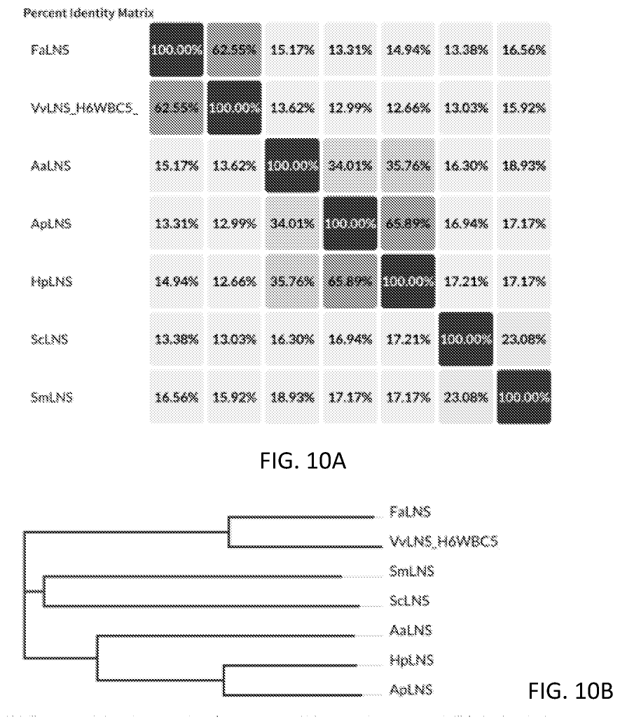

In addition, the inventors of the present disclosure observed that nerolidol produced in all the strains of the present disclosure is 100% trans-nerolidol, whereas synthetic nerolidol consists of both cis- and trans-nerolidol in an estimated ratio of 42:58, respectively (FIG. 2B). The two isomers cis- and trans-nerolidol have very similar mass spectra but different retention indices (FIG. 11). The retention index of trans-nerolidol is 1564 (non-polar column such as DB5) which is larger than that of cis-nerolidol that is 1544.

### Pathway Optimization

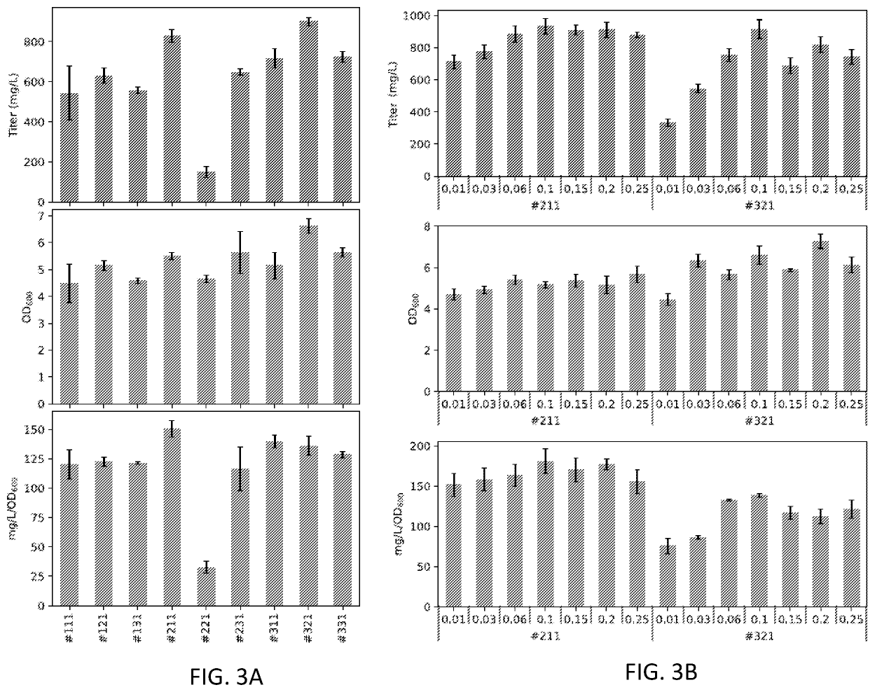

Next, the inventors of the present disclosure focused on FaLNS and optimized the upstream mevalonate pathway (modules 1 and 2) by promoter engineering. Here, the inventors of the present disclosure used three T7 promoter variants, TM1/2/3. The transcriptional efficiencies of TM1, TM2, and TM3 promoters are about 92%, 37%, and 16% of that of the wildtype T7 promoter, respectively. T7 and TM1/2/3 promoters have been successfully applied in modular metabolic engineering and MHP methods to produce various high-value metabolites by systematically balancing the biosynthetic pathway. FIGS. 3A and 3B show optimization of the biosynthetic pathway and inducer concentrations. Among the nine constructs the inventors of the present disclosure tested, the two strains #211 and #321 produced the highest titres of nerolidol (FIG. 3A). Details of the nine strains are shown in Tables 3. Superficially, strain #211 used relatively stronger promoters TM2 and TM1 to regulate modules 1 and 2, respectively. Strain #321 used relatively weaker promoters TM3 and TM2 to control the two modules, respectively. Module 3 in the two strains was regulated by the same TM1 promoter. The inventors of the present disclosure further tuned the IPTG concentration for the two strains #211 and #321. The highest titres of the two strains were similar, ˜910 mg/L (FIG. 3B). However, strain #211 had a wider optimal IPTG range of 0.06-0.25 mM for nerolidol production; hence, the inventors of the present disclosure selected #211 for future work. In contrast, strain #321 was more sensitive to IPTG and had the highest yield of nerolidol only with 0.1 mM IPTG. Lower or higher concentrations of IPTG resulted in noticeably lower production of nerolidol in #321 strain. The OD₆₀₀ values were also different for the two strains: the values of strain #321 varied between 4.4 and 7.3, while those of strain #211 were between 4.7 and 5.7 (FIG. 3B). Due to the relatively lower OD₆₀₀ values, the highest specific yield of #211 (˜181 mg/L/OD₆₀₀) was relatively higher than that of #321 (˜138 mg/L/OD₆₀₀, FIG. 3B).

### Medium Optimization

Next, the inventors of the present disclosure tested two types of chemically defined media (CDM). The first one is IPTG-induced CDM (type 1), with 10-12.7 g/L glucose as the only carbon source. The second one is lactose automatically induced CDM, with 2 g/L glucose, 8 g/L glycerol, and 15 mM lactose (type 2). Like glucose, glycerol is another abundant carbon source, mainly produced as the byproduct of biodiesel. The price of crude glycerol from biodiesel is $198-220/ton, about half that of glucose (˜$400/ton). In addition, glucose oversupply (>20 g/L) in E. coli typically leads to overproduction of acetic acid which limits cell growth and bioproduction of secondary metabolites. In contrast, glycerol oversupply does not lead to overproduction of acetic acid. Hence, the use of glycerol can simplify the fermentation process and potentially have higher carbon yields.

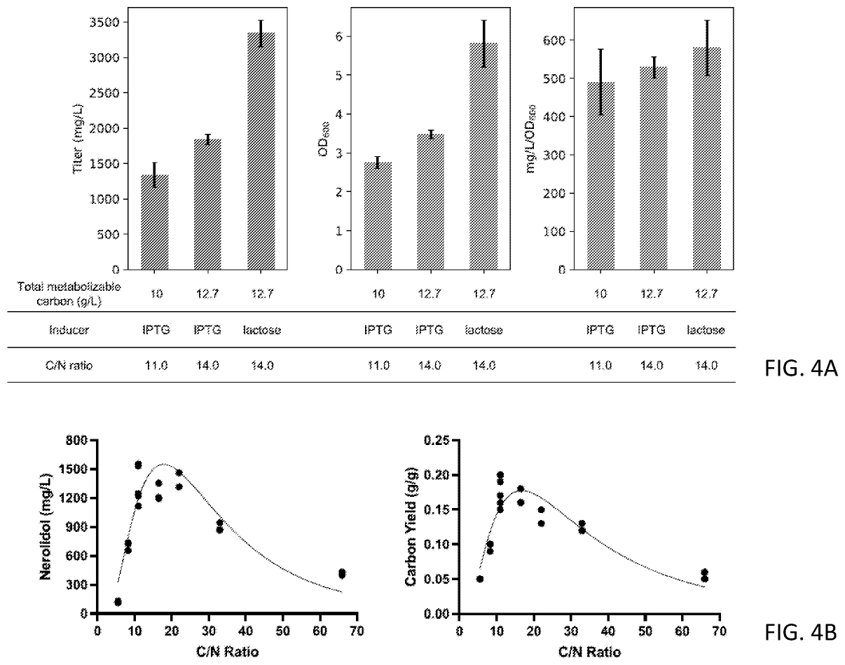

FIGS. 4A and 4B shows comparison of different carbon sources, C/N ratios, and inducers. Lactose functions as an inducer and an additional carbon source in type 2 medium. However, the MG1655 strain used in this study can metabolize only half the amount of lactose. In E. coli, lactose is first cleaved by β-galactosidase (lacZ gene) to galactose and glucose. The MG1655 strain can assimilate only glucose but not galactose. Hence, in type 2 medium, the total metabolizable carbon is about 12.7 g/L. For strain #211a (strain #211 with tnaA deletion, Table 3), the nerolidol titres in lactose media (3.3 g/L) were about >80% higher than those in IPTG media (1.8 g/L) with the same amount of metabolizable carbon (FIG. 4A). Also, the OD₆₀₀ values and specific yields were also relatively higher in lactose media than those in IPTG media. The carbon yield (0.26 g nerolidol/g carbon source) was 86% higher in lactose media than that in IPTG media (0.14 g nerolidol/g carbon source). The highest yield reached 26.2% (g/g), >90% of the theoretic yield.

In addition, the inventors of the present disclosure observed that 27% increase in glucose in type 1 media resulted in 37% increase in nerolidol titres (FIG. 4A). The additional increase could be due to the C/N molar ratio change in the media, in which a C/N ratio of 14 was more favourable for nerolidol biosynthesis than a C/N ratio of 11. To test this, the inventors of the present disclosure further designed an experiment varying the concentrations of glucose (C) and ammonium sulfate (N). As expected, the C/N ratio did have impact on nerolidol production (FIG. 4B), with an optimal range of C/N ratio at 11-20.

### Genome Editing for Nerolidol Bioproduction

Next, the inventors of the present disclosure targeted the E. coli genome by blocking the formation of potential byproducts and several other strategies. The first set of targets contains six genes involved in mixed acid fermentation (FIG. 1): poxB, pyruvate dehydrogenase that produces acetate from pyruvate; pflB, formate acetyltransferase 1 that produces formate from pyruvate; IdhA, D-lactate dehydrogenase that produces lactate from pyruvate; ackA, acetate kinase that produces acetate from acetyl phosphate; pta, phosphate acetyltransferase that converts acetyl-CoA to acetyl phosphate (ackA and pta were deleted together); and adhE, aldehyde/alcohol dehydrogenase that produces ethanol from acetyl-CoA. The deletion of the first group could block byproduct formation (e.g., acetate, formate, and lactate), thus channelling more carbon flux toward the terpene pathway thereby increasing the production of nerolidol. The second group consisted of only tnaA (tryptophanase), which catalyses the formation of indole and pyruvate from tryptophan or pyruvate from cysteine (FIG. 1). The reason to delete tnaA was to prevent the formation of indole, which gives the unpleasant smell of the E. coli culture. The last group is zapB, the cell division factor. The deletion of zapB could lead to the delayed cell division and thus elongated cells, which was known to enhance the storage of intracellular metabolites. In the application of the present disclosure, the inventors of the present disclosure found that nerolidol was mainly intracellular (>90%) in E. coli in single-phase fermentation.

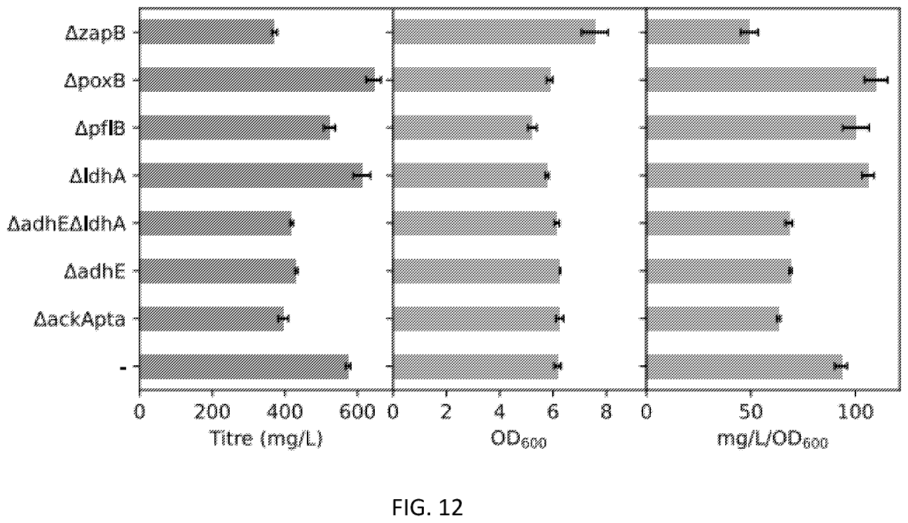

The inventors of the present disclosure first tested the strain in IPTG-induction media, the deletion of poxB and IdhA resulted in about 12% and 7% increase in nerolidol production, respectively (FIG. 15). However, the deletion of ackA-pta, adhE, pflB, and zapB reduced the nerolidol yield. The double deletion of adhE and IdhA also reduced the nerolidol production. For biomass, only zapB mutation increased OD₆₀₀ values which could be the outcome of cell elongation that affected light scattering rather than more cells. The pflB and poxB deletion resulted in less biomass as compared to that of the parental strain. Hence, the specific yields (mg/L/OD₆₀₀) of nerolidol were 18%, 8%, and 14% higher in the poxB, pflB, and IdhA deleted strain than that of the parental strain (#211), respectively.

Next, the inventors of the present disclosure compared the single and multiple deletion in lactose-induction media. The inventors of the present disclosure observed that most strains produced higher yields of nerolidol (FIG. 5) than that of the parental strain (#211). Particularly, the deletion of IdhA, pflB, poxB, zapB, ackA-ptaA, and adhE-IdhA resulted in 15%, 14%, 6%, 10%, 14%, and 9% increase in nerolidol titres, respectively. The triple deletion strain (ΔadhEΔldhAΔpflB) increased nerolidol titre by 16%. In contrast, the mutants (ΔadhEΔldhAΔpoxB and ΔadhEΔldhAΔackApta) had similar titers of nerolidol as compared to #211. However, the mutants (ΔadhEΔldhAΔ-poxBΔpflB and ΔadhEΔldhAΔpoxBΔpflBΔackApta) suffered from poor growth, with the final OD₆₀₀ at 0.7-0.8. Hence, the nerolidol production was very low, ˜1 mg/L.

### TnaA Deletion and Lactose Optimization

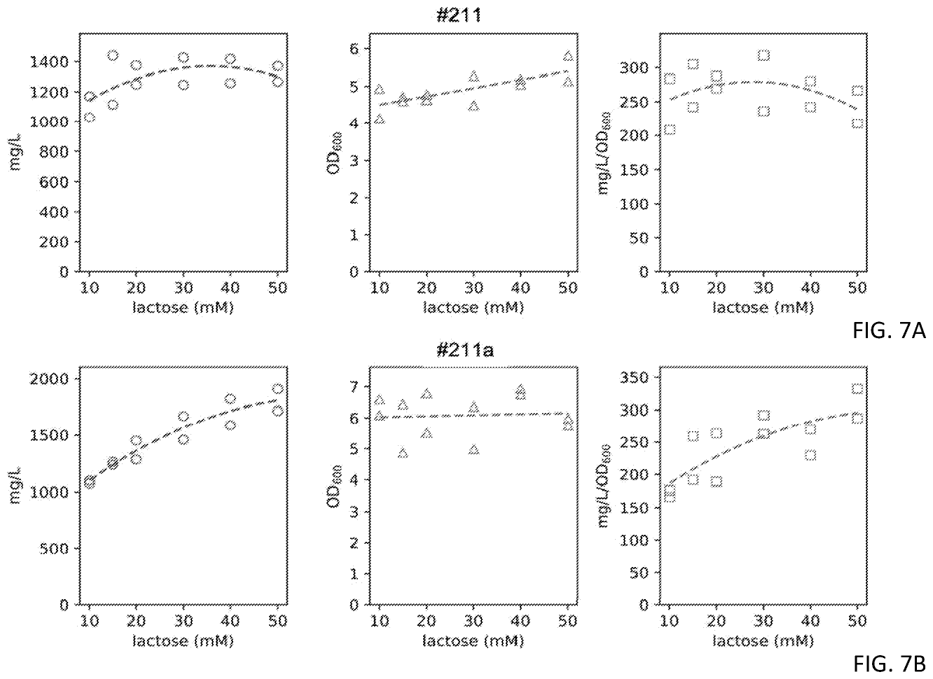

Next, the inventors of the present disclosure checked the effect of lactose concentration on the two strains, #211 and #211a (#211 with tnaA deletion). FIGS. 7A and 7B show the effect of tnaA deletion and lactose concentration on nerolidol bioproduction. The inventors of the present disclosure found that the nerolidol titre of #211 was plateaued at 1.3 g/L with 30 mM of lactose (FIG. 7A). Further increase in lactose only resulted in slightly higher biomass but not nerolidol production. In contrast, the nerolidol titre of #211a strain continuously increased as lactose concentration increased (FIG. 7B). The nerolidol titre reached about 1.8 g/L with 50 mM lactose from 1.1 g/L with 10 mM lactose. The specific yields had similar trends as titres of nerolidol for the two strains.

### Comparison of Different Extractants

Next, the inventors of the present disclosure compared the use of plant oils such as sunflower oil with the commonly used dodecane as the extractant in bioreactors. The data of the present disclosure indicated the use of sunflower oil led to 20% and 34% higher nerolidol production and biomass than that of dodecane, respectively (FIG. 8). The benefit could be attributed to the boosting effect of some nutrients (e.g., vitamins and fatty acids) in sunflower oil on E. coli cell growth.

### Fed-Batch Fermentation of Nerolidol

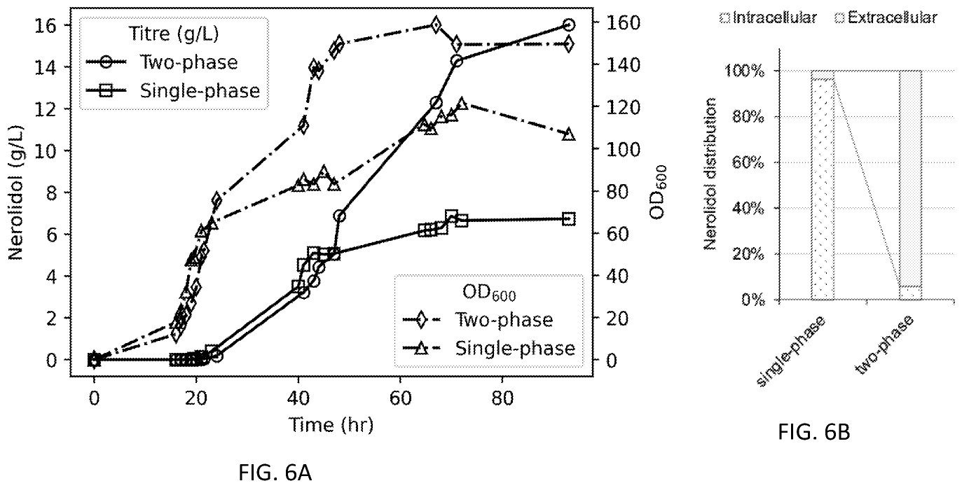

The inventors of the present disclosure then tested strain #211a in 5 L bioreactors in a fed-batch process. The inventors of the present disclosure studied both single-phase fermentation and two-phase fermentation (with the supplementation of dodecane) (FIGS. 6A and 6B). In single-phase, the strain of the present disclosure produced ˜7 g/L nerolidol in 70 h, with >96% as intracellular (˜6.8 g/L) and only 0.14% as extracellular nerolidol (˜10 mg/L) in aqueous media (FIG. 6A, 6B). In addition, about >3.4% of nerolidol (>241 mg/L) was evaporated in the exhausted gas, which the inventors of the present disclosure captured by submerging exit tubing in dodecane. In two-phase fermentation, the strain produced >16 g/L nerolidol in 93 h (FIG. 6A), which is 50-190% times higher than current best achievements. In contrast to single phase fermentation, in a two-phase bioreactor, 94% nerolidol was extracellular mainly in organic layer, and intracellular nerolidol was about 6% (FIG. 6B). In both processes, the inventors of the present disclosure observed that OD₆₀₀ plateaued at 60-70 h, the inventors of the present disclosure hypothesized that the accumulation of nerolidol and/or other metabolites (such as acetic acid) imposed stress on cells and prevented them from further growth. This observation is consistent with other terpenoid bioproduction (e.g., viridiflorol and amorphadiene) in E. coli.

LNSs can accept both geranyl diphosphate (GPP) and farnesyl diphosphate (FPP) as substrates to produce linalool and nerolidol, respectively. Here, the inventors of the present disclosure did not detect any linalool in GC/MS analysis of the samples of the present disclosure. This could be likely due to microbes including E. coli do not have endogenous GPP synthases; therefore, the GPP availability in microbes is low. According to the in vitro study, the bacterial ScLNS has the highest Kcat; however, when expressed in E. coli, the strawberry LNS gave the highest yield of nerolidol, indicating the differences between in vitro and in vivo experiments. This shows that the nerolidol synthase in the biosynthetic pathway is one of the limiting factors. This is consistent with the previous study of this present disclosure that viridiflorol (another sesquiterpene) synthase is the key limiting factor in producing viridiflorol from glucose. The viridiflorol synthase activity was increased by protein engineering, which enabled a near-theoretical-yield of viridiflorol bioproduction. Last year another study in the art also used the same FaLNS to produce nerolidol in Y. lipolytica. The study had similar observation that FaLNS was the rate-limiting step and identified the key mutation G498Q, which greatly increased the activity by threefold than that of wild-type FaLNS. In the future, the inventors of the present disclosure will explore protein engineering of FaLNS to further improve the nerolidol bioproduction.

Here, the inventors of the present disclosure observed that lactose-induction media led to >80% higher yield than that in IPTG media (26% versus 14% g/g), indicating that the carbon sources (glucose or glycerol) are critical for nerolidol bioproduction. Furthermore, the inventors of the present disclosure observed that the optimal C/N ratio for nerolidol biosynthesis was between 11 and 20. Also, the inventors of the present disclosure observed that media and inducers affect the performance of genomic editing strategies. Some deletion mutants (e.g., ΔadhE, ΔpflB, and ΔackApta) behaved differently in IPTG-induction and lactose-induction media. Therefore, to obtain the best performance, genomic editing and media optimization strategies should be combined.

In small-scale tubes and flasks, the inventors of the present disclosure tested the strains of the present disclosure in two phase fermentation. About 20% (v/v) of dodecane was supplemented on top of cell culture to capture nerolidol. However, the dodecane supplementation resulted in variations in the biomass measurement. The OD₆₀₀ values were not always very consistent and fluctuated from 3 to 6, which also contributed to the variations in specific yields of nerolidol. To avoid such variation, in the future, the inventors of the present disclosure will use dry cell weight instead of OD₆₀₀. In addition, as compared in bioreactors, two phase extractive fermentation outperformed the single-phase fermentation for nerolidol by over two times possibly because in situ extraction drives the reactions toward nerolidol production and overcomes the intracellular storage limitation. Hence, two-phase fermentation is recommended for nerolidol bioproduction.

Although the inventors of the present disclosure have identified that lactose auto-induction media gave higher nerolidol yields, the inventors of the present disclosure have not developed the fed-batch bioprocess with the auto-induction media. The initial trial in bioreactors did not produce the expected results. As such, the inventors of the present disclosure used the protocol that used glucose and IPTG. In flasks, the nerolidol yield is about 14% in glucose only media; however, the yield dropped to 9% in bioreactors, indicating that there is room for future bioprocess optimization. Even so, the titre and productivities (2.3-4.5 times) of the present disclosure are much higher than all the previous results in the art. Also, the media of the present disclosure are chemically defined ones and do not contain yeast extract, peptone, tryptone, or vitamins, which typically have batch-to-batch variations. Therefore, the media of the present disclosure are much less costly and more consistent. In the future, the inventors of the present disclosure aim to develop the bioprocess based on lactose autoinduction media, which renders even higher carbon yields and titres.

The inventors of the present disclosure have developed microbial strains to overproduce nerolidol. The metabolic engineering and bioprocess strategies are proven to be effective and have synergic effects in improving the yields of nerolidol by identifying the best enzymes and their combinations, balancing the metabolic pathways, supplying carbon precursors, and blocking the competitive pathways that lead to by-products (e.g., acetate). The present disclosure demonstrated microbial production of nerolidol and inspire the production of other high-value metabolites.

### DETAILED DESCRIPTION OF FIGURES

Example embodiments of the disclosure will be better understood and readily apparent to one of ordinary skill in the art from the following discussions and if applicable, in conjunction with the figures. Example embodiments are not necessarily mutually exclusive as some may be combined with one or more embodiments to form new exemplary embodiments. The example embodiments should not be construed as limiting the scope of the disclosure.

FIG. 1 show a schematic diagram of the biosynthetic pathway of carotenoid glucosides. The biosynthetic pathway: module 1 AHT, including atoB, hmgS, and thmgR; module 2 MPPI, including mevk, pmk, pmd, and idi; and module 3 FL, including fpps and LNS. Dashed arrow indicates multiple enzymatic steps. Abbreviation for the compounds: HMG-COA, 3-hydroxy-3-methyl-glutaryl-coenzyme A; MVA, mevalonate; MVAP, phosphomevalonate; MVAPP, diphosphomevalonate; IPP, isopentenyl pyrophosphate; DMAPP, dimethylallyl pyrophosphate; and FPP, farnesyl pyrophosphate. The genes expressed encode the following enzymes: atoB, acetoacetyl-CoA thiolase; hmgS, HMG-COA synthase; thmgR, truncated HMG-COA reductase; mevk, mevalonate kinase; pmk, phosphomevalonate kinase; pmd, mevalonate pyrophosphate decarboxylase; idi, IPP isomerase; fpps, FPP synthase; LNS, nerolidol synthase; poxB, pyruvate dehydrogenase; pflB, formate acetyltransferase 1; IdhA, D-lactate dehydrogenase; ackA, acetate kinase; pta, phosphate acetyltransferase; and adhE, aldehyde/alcohol dehydrogenase; tnaA, tryptophanase; and zapB, cell division factor. Enzymes are shown in grey color.

FIG. 2A shows graphs with titre, OD₆₀₀, and specific yields of various strains expressing different nerolidol synthases. Enzyme information is listed in Table 1. Error bars, mean±s.d., n=3-6.

FIG. 2B shows GC-MS chromatograms of the samples of the present disclosure and an authentic nerolidol standard.

FIG. 3A shows graphs of pathway optimization with promoters of different strengths. The digits in the strain names represent the promoter used for each module. For example, strain “#231” refers to the modules 1/2/3 in the strain are controlled by TM2, TM3, and TM1, respectively. More details are shown in Tables 3 and 4.

FIG. 3B shows graphs with titre, OD₆₀₀, and specific yields of the top two strains #211 and #321 with different concentrations (0.01-0.25 mM) of IPTG. Error bars, mean±s.d., n=3.

FIG. 4A shows graphs with titre, OD₆₀₀, and specific yields of the strain #211a in two types of media, (1) 10-12.7 g/L glucose induced by 0.1 mM IPTG and (2) 2 g/L glucose and 8 g/L glycerol induced by 15 mM lactose. Error bars, mean+s.d., n=3-6.

FIG. 4B shows plots with nerolidol titres and yields with various C/N ratios.

FIG. 5 shows graphs of the effect of single or multiple gene deletion on nerolidol bioproduction. All the strains were grown in defined media with 2 g/L glucose and 8 g/L glycerol and induced by 15 mM lactose. “-” refers to the control strain (#211) without the deletion of any of these seven genes, ackA, adhE, IdhA, pta, poxB, pflB, and zapB. Error bars, mean+s.d., n=2.

FIG. 6A shows plots with time course of nerolidol titres and OD₆₀₀. Strain #211a was used in chemically defined medium with glucose as the carbon source and induced by 0.1 mM IPTG when OD₆₀₀ reached ˜50.

FIG. 6B shows bar chart of nerolidol distribution in single-phase (mainly intracellularly) and two-phase (mainly extracellularly in organic layer) fermentations.

FIG. 7A shows plots of strain #211 grown in the chemically defined medium with 2 g/L glucose and 8 g/L glycerol and induced by 15 mM lactose.

FIG. 7B shows plots of Strain #211a (or #211ΔtnaA) grown in the chemically defined medium with 2 g/L glucose and 8 g/L glycerol and induced by 15 mM lactose.

FIG. 8 shows charts with the comparison of dodecane and sunflower oil as the extractant. The strains were grown in defined media with 2 g/L glucose and 8 g/L glycerol and induced by 15 mM lactose. Here, 20% of dodecane or sunflower oil was used during fermentation to capture the nerolidol.

FIG. 9 shows sequence alignment of various nerolidol synthases. The alignment is done by Clustal Omega v1.2.4. The motif residues in DDxxD, NDSE and WxxxxxRY (only conserved in microbial nerolidol synthases) were highlighted in grey. Other information about the enzymes is in Table 1.

FIG. 10A shows a percent identity matrix of the seven nerolidol synthases.

FIG. 10B shows the phylogenetic tree of the seven nerolidol synthases, which is generated with the online UniProt alignment tool.

FIG. 11 shows the mass spectra of the sample of the present disclosure and authentic standards. Reference mass spectra are provided from National Institute of Standards and Technology (NIST) library.

FIG. 12 shows graphs of the effect of single or double gene deletion on nerolidol bioproduction. The details for the genes ackA, adhE, IdhA, pta, poxB and pflB are listed in FIG. 1. In addition, zapB, the cell division factor, was also tested. “-” refers to the control strain (#211) without the deletion of any of the 7 genes. All the strains were grown in the chemically defined medium with 10 g/L glucose and induced by 0.1 mM IPTG. Error bars, mean±s.d., n=2.

### APPLICATIONS

Embodiments as disclosed herein provide a recombinant cell comprising a plasmid expressing terpenoid synthase gene and one or more modification to the gene of the recombinant cell, a recombinant cell for use in biosynthesis, and a method of producing isoprenoid/terpenoid.

Advantageously, the present invention uses strawberry nerolidol synthase in Escherichia coli in the biosynthesis of isoprenoid. The strawberry nerolidol synthase produced the highest nerolidol titre and nerolidol yields as compared to nerolidol synthase from other microbial hosts (such as fungal, bacteria).

More advantageously, the present invention includes the deletion and/or insertion of single and/or multiple genes of the recombinant cell that have been found to surprisingly improve the biosynthesis of isoprenoid.

Even more advantageously, the present invention uses the combination of various carbon sources (such as glucose, glycerol, lactose) that the inventors have found to improve isoprenoid biosynthesis. For example, the inventors found that the use of combination of various carbon sources with lactose-induction media can lead to >80% higher nerolidol production yield than that in IPTG media.

Even more advantageously, the present inventors found that the use of sunflower oil as extractant in bioreactor can lead to 20% and 34% higher nerolidol production and biomass than that of a commonly used extractant dodecane.

Even more advantageously, the present invention uses bioprocess conditions (such as single-phase and two-phase with organics) for isoprenoid biosynthesis. In two-phase fermentation, the inventors found that the strain produced nerolidol titre that is 50-190% times higher than studies in the art. In addition, 94% nerolidol are extracellular compared to single-phase fermentation which has only 0.14% extracellular nerolidol.

Even more advantageously, the present invention can be used as the raw material to synthesize high value pharmaceuticals (such as teprenone, α-sinensal and 4-acetylantroquinolol B.

Even more advantageously, the present invention can be used in pharmaceutical products in anti-ulcer, anti-tumour, anti-inflammatory, antioxidant, anti-fungal purposes.

Even more advantageously, the present invention can be used in personal care and cosmetic products including deodorants, lotion, perfumes, mouthwash and creams.

Even more advantageously, the present invention can be used in food flavours and food preservatives.

Even more advantageously, the present invention can be used in pesticides, primarily as an insect repellent.

Even more advantageously, the present invention can be used to further improve the product yields and titres of isoprenoid production by developing next generation of microbial strains.

Even more advantageously, the present invention can be used to further improve the bioprocess, especially on fermentation.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A recombinant cell comprising a plasmid expressing a terpenoid synthase gene and one or more modification to the gene of the recombinant cell.

2. The recombinant cell according to claim 1, wherein the terpenoid synthase is a sesquiterpenoid synthase.

3. The recombinant cell according to claim 1 or 2, wherein the sesquiterpenoid synthase is nerolidol synthase.

4. The recombinant cell according to any one of the preceding claims, wherein the terpenoid synthase is selected from the group consisting of fungi, plants and/or bacteria.

5. The recombinant cell according to any one of the preceding claims, wherein the terpenoid synthase is from any one selected from the group consisting of Fragaria ananassa (Fa)-strawberry, Vitis vinifera-grape, Selaginella moellendorffii (Sm)-Spikemoss, Celastrus angulatus Maxim, Maze Zea mays, Abrus precatorius, Acer yangbiense, Actinidia chinensis, Aegilops tauschii, Ageratina Adenophora, Albizia julibrissin, Ananas comosus, Antirrhinum majus, Apostasia shenzhenica, Aquilegia coerulea, Arabidopsis thaliana, Arabis alpine, Arachis duranensis, Arachis hypogaea, Artemisia annua, Beta vulgaris, Cajanus cajan, Camellia sinensis, Cannabis sativa, Capsicum annuum, Capsicum baccatum, Capsicum chinense, Carpinus fangiana, Castanea mollissima, Chengiopanax sciadophylloides, Chenopodium quinoa, Cicer arietinum, Citrus clementina, Citrus sinensis, Citrus unshiu, Coffea arabica, Coffea canephora, Coleus amboinicus, Coptis chinensis, Cucumis sativus, Cucurbita maxima, Cucurbita moschata, Davidia involucrate, Dorcoceras hygrometricum, Ensete ventricosum, Erythranthe guttata, Eucalyptus grandis, Fagus sylvatica, Ficus carica, Folsomia candida, Fragaria vesca, Glycine max, Glycine soja, Gossypium australe, Helianthus annuus, Herrania umbratical, Hevea brasiliensis, Hibiscus syriacus, Jasminum sambac, Jatropha curcas, Juglans regia, Kalanchoe fedtschenkoi, Kingdonia uniflora, Lactuca saligna, Lavandula angustifolia, Lupinus albus, Lupinus angustifolius, Malus baccata, Malus domestica, Manihot esculenta, Medicago sativa, Medicago truncatula, Microthlaspi erraticum, Mikania micrantha, Momordica charantia, Morella rubra, Morus notabilis, Mucuna pruriens, Nelumbo nucifera, Nicotiana attenuate, Nicotiana tabacum, Nyssa sinensis, Ocimum basilicum, Olea europaea subsp. Europaea, Oryza brachyantha, Panicum miliaceum, Phaseolus angularis, Phaseolus vulgaris, Phyllostachys edulis, Populus alba, Populus nigra, Populus trichocarpa, Prunus armeniaca, Prunus avium, Prunus campanulate, Prunus dulcis, Prunus persica, Punica granatum, Pyrus ussuriensis x Pyrus communis, Quercus lobata, Rhamnella rubrinervis, Rosa chinensis, Salix brachista, Salix dunnii, Salix suchowensis, Scoparia dulcis, Selaginella moellendorffii, Senna tora, Solanum lycopersicum, Spinacia oleracea, Striga asiatica, Tanacetum cinerariifolium, Tetracentron sinense, Theobroma cacao, Trifolium medium, Trifolium pratense, Trifolium subterraneum, Tripterygium wilfordii, Triticum Urartu, Vigna angularis var. angularis, Vigna radiata var. radiata, Zingiber officinale, and Ziziphus jujuba.

6. The recombinant cell according to any one of the preceding claims, wherein the terpenoid synthase is a strawberry nerolidol synthase (FaLNS).

7. The recombinant cell according to any one of the preceding claims, wherein the recombinant cell comprises one or more modifications to the gene of the recombinant cell, optionally the modification to the gene of the recombinant cell comprises one or more insertion and/or one or more deletion of the recombinant cell.

8. The recombinant cell according to any one of the preceding claims, wherein the one or more modification to the gene of the recombinant cell comprises genes involved in the formation of by-products.

9. The recombinant cell according to any one of the preceding claims, wherein the one or more modification to the gene of the recombinant cell comprises genes from the mixed acid fermentation pathway and/or lyase family.

10. The recombinant cell according to any one of the preceding claims, wherein the modification to the gene of the recombinant cell is to one or more genes selected from the group consisting of lactose inhibitor (lacI), T7 RNA polymerase (T7RP), aroA, aroB, aroC, aroAB, aroBC, aroAC, aroABC, ackA, pta, ackA-pta, IdhA, pflB, poxB, adhE, adhE-pta, serC, frdABCD, eutD, adhE-IdhA, adhE-IdhA-pflB, adhE-IdhA-poxB, adhE-IdhA-ackA-pta, adhE-IdhA-poxB-pfIB, poxB-pflB-ackA-pta, adhE-IdhA-poxB-pfIB-ackA-pta, tnaA, zapB, and combinations thereof.

11. The recombinant cell according to any one of the preceding claims, wherein the one or more genes that are modified from the recombinant cell comprises the combination of ackA-pta, IdhA, pflB, poxB, and/or tnaA.

12. The recombinant cell according to any one of the preceding claims, wherein the recombinant cell comprises an insertion of lactose inhibitor and/or T7 RNA polymerase between the ybhB and ybhC gene.

13. The recombinant cell according to any one of the preceding claims, wherein the recombinant cell comprises a deletion of aroA, aroB, aroC, aroAB, aroBC, aroAC, aroABC, ackA, pta, ackA-pta, IdhA, pflB, poxB, adhE, adhE-pta, adhE-IdhA, adhE-IdhA-pfIB, adhE-IdhA-poxB, adhE-IdhA-ackA-pta, adhE-IdhA-poxB-pflB, poxB-pfIB-ackA-pta, adhE-IdhA-poxB-pfIB-ackA-pta, tnaA, and/or zapB.

14. The recombinant cell according to any one of the preceding claims, wherein the recombinant cell carries plasmids expressing genes of a biosynthetic pathway.

15. The recombinant cell according to any one of the preceding claims, wherein the biosynthetic pathway comprises the nerolidol biosynthetic pathway and/or mevalonate pathway.

16. The recombinant cell according to any one of the preceding claims, wherein the recombinant cell carries plasmid expressing genes of the nerolidol biosynthetic pathway and/or mevalonate pathway comprising hmgS, atoB, truncated HMG-COA reductase (thmgR), mevk, pmk, pmd, and/or idi.

17. The recombinant cell according to any one of the preceding claims, wherein the recombinant cell comprises one or more plasmids with a combination of strong or weak promoters.

18. The recombinant cell according to any one of the preceding claims, wherein the recombinant cell comprises a combination of T7, TM1, TM2 and/or TM3 promoters.

19. The recombinant cell according to any one of the preceding claims, wherein the recombinant cell comprises one or more modifications selected from the group consisting of
i) a deletion of ackA and pta,
ii) carrying plasmids expressing one or more genes selected from the group consisting of HMG-COA synthase (hmgS), acetoacetyl-CoA thiolase (atoB), HMG-COA reductase (hmgR), aroC with T7, TM1, TM2 and/or TM3 promoter; mevalonate kinase (mevK), phosphomevalonate kinase (pmk), mevalonate pyrophosphate decarboxylase (pmd), IPP isomerase (idi), aroB with T7, TM1, TM2 and/or TM3 promoter; and/or
iii) nerolidol synthase (LNS), fpps (ispA), aroA with T7, TM1, TM2 and/or TM3 promoter;
iv) a deletion of IdhA,
v) carrying plasmids expressing hmgS, atoB, hmgR, aroC with T7, TM1, TM2 and/or TM3 promoter; mevk, pmk, pmd, idi, aroB with T7, TM1, TM2 and/or TM3 promoter; and/or
vi) LNS, ispA, aroA with T7, TM1, TM2, and/or TM3 promoter;
vii) a deletion of pflB,
viii) carrying plasmids expressing hmgS, atoB, hmgR, aroC with T7, TM1, TM2 and/or TM3 promoter; mevk, pmk, pmd, idi, aroB with T7, TM1, TM2 and/or TM3 promoter;
and/or
ix) LNS, ispA, aroA with T7, TM1, TM2 and/or TM3 promoter;
x) a deletion of poxB,
xi) carrying plasmids expressing hmgS, atoB, hmgR, aroC with T7, TM1, TM2 and/or TM3 promoter; mevk, pmk, pmd, idi, aroB with T7, TM1, TM2 and/or TM3 promoter;
and/or
xii) LNS, ispA, aroA with T7, TM1, TM2 and/or TM3 promoter;
xiii) a deletion of tnaA,
xiv) carrying plasmids expressing hmgS, atoB, hmgR, aroC genes with T7, TM1, TM2 and/or TM3 promoter; mevk, pmk, pmd, idi, aroB genes with T7, TM1, TM2 and/or TM3 promoter; and/or
xv) LNS, ispA, aroA with T7, TM1, TM2 and/or TM3 promoter;
xvi) a deletion of adhE,
xvii) carrying plasmids expressing hmgS, atoB, hmgR, aroC genes with T7, TM1, TM2 and/or TM3 promoter; mevk, pmk, pmd, idi, aroB genes with T7, TM1, TM2 and/or TM3 promoter; and/or
xviii) LNS, ispA, aroA with T7, TM1, TM2 and/or TM3 promoter;
xix) a deletion of zapB,
xx) carrying plasmids expressing hmgS, atoB, hmgR, aroC genes with T7, TM1, TM2 and/or TM3 promoter; mevk, pmk, pmd, idi, aroB genes with T7, TM1, TM2 and/or TM3 promoter; and/or
xi) LNS, ispA, aroA with T7, TM1, TM2 and/or TM3 promoter.

20. The recombinant cell according to any one of the preceding claims, wherein the recombinant cell is a microbial cell selected from the group consisting of a bacterial cell, a fungal cell, a plant cell, and an algae cell.

21. The recombinant cell according to any one of the preceding claims, wherein the microbial cell is a bacterial cell, optionally the microbial cell is Escherichia coli.

22. A recombinant cell according to any one of the preceding claims for use in biosynthesis.

23. A method of producing isoprenoid, where in the method comprises culturing the recombinant cell according to any one of the preceding claims.

24. The method according to claim 23, wherein the method comprises culturing the recombinant cell in a medium comprising a carbon source with glucose, glycerol and/or lactose.

25. The method according to claim 23 or 24, wherein the method comprises culturing the recombinant cell in a medium with an extractant that comprises a plant oil.

26. The method according to any one of claims 23 to 25, wherein the extractant comprises a sunflower oil.

27. The method according to any one of claims 23 to 26, wherein the method comprises culturing the recombinant cell with a bioprocess comprising fed-batch fermentation with single-phase fermentation and/or two-phase fermentation.
